SEQUENCE LISTING (34 sequences)

SEQ ID NO: 1 - protein, 13 aa, Artificial Sequence
  Synthetic peptide
  MISC_FEATURE (3)..(3): Xaa is Ahp
  MISC_FEATURE (10)..(10): Xaa is Bph
  MISC_FEATURE (11)..(11): Xaa is Ahp
ARXYHDGVLXXDC

SEQ ID NO: 2 - protein, 12 aa, Artificial Sequence
  Synthetic Peptide
  MISC_FEATURE (4)..(4): Xaa is MePhe
  MISC_FEATURE (8)..(8): Xaa is Bph
  MISC_FEATURE (11)..(11): Xaa is Ahp
ALHXVLPXVWVC

SEQ ID NO: 3 - protein, 13 aa, Artificial Sequence
  Synthetic Peptide
  MISC_FEATURE (3)..(3): Xaa is Ahp
  MISC_FEATURE (10)..(10): Xaa is Bph
  MISC_FEATURE (11)..(11): Xaa is Ahp
ARXYHDSVLXXDC

SEQ ID NO: 4 - protein, 13 aa, Artificial Sequence
  Synthetic Peptide
  MISC_FEATURE (3)..(3): Xaa is Ahp
  MISC_FEATURE (10)..(10): Xaa is Bph
  MISC_FEATURE (11)..(11): Xaa is Ahp
ARXYHDQVLXXDC

SEQ ID NO: 5 - protein, 13 aa, Artificial Sequence
  Synthetic Peptide
  MISC_FEATURE (3)..(3): Xaa is Ahp
  MISC_FEATURE (10)..(10): Xaa is Bph
  MISC_FEATURE (11)..(11): Xaa is Ahp
ARXYHDNVLXXDC

SEQ ID NO: 6 - protein, 13 aa, Artificial Sequence
  Synthetic Peptide
  MISC_FEATURE (3)..(3): Xaa is Ahp
  MISC_FEATURE (7)..(7): Xaa is MetO2
  MISC_FEATURE (10)..(10): Xaa is Bph
  MISC_FEATURE (11)..(11): Xaa is Ahp
ARXYHDXVLXXDC

SEQ ID NO: 7 - protein, 13 aa, Artificial Sequence
  Synthetic Peptide
  MISC_FEATURE (3)..(3): Xaa is Ahp
  MISC_FEATURE (10)..(10): Xaa is Bph
  MISC_FEATURE (11)..(11): Xaa is Ahp
ARXYHDGVTXXDC

SEQ ID NO: 8 - protein, 13 aa, Artificial Sequence
  Synthetic Peptide
  MISC_FEATURE (3)..(3): Xaa is Ahp
  MISC_FEATURE (9)..(9): Xaa is Hse
  MISC_FEATURE (10)..(10): Xaa is Bph
  MISC_FEATURE (11)..(11): Xaa is Ahp
ARXYHDGVXXXDC

SEQ ID NO: 9 - protein, 13 aa, Artificial Sequence
  Synthetic Peptide
  MISC_FEATURE (3)..(3): Xaa is Ahp
  MISC_FEATURE (9)..(9): Xaa is MetO2
  MISC_FEATURE (10)..(10): Xaa is Bph
  MISC_FEATURE (11)..(11): Xaa is Ahp
ARXYHDGVXXXDC

SEQ ID NO: 10 - protein, 13 aa, Artificial Sequence
  Synthetic Peptide
  MISC_FEATURE (3)..(3): Xaa is Ahp
  MISC_FEATURE (10)..(10): Xaa is Bph
  MISC_FEATURE (11)..(11): Xaa is Ahp
ARXYHDEVLXXDC

SEQ ID NO: 11 - protein, 13 aa, Artificial Sequence
  Synthetic Peptide
  MISC_FEATURE (3)..(3): Xaa is Ahp
  MISC_FEATURE (10)..(10): Xaa is Bph
  MISC_FEATURE (11)..(11): Xaa is Ahp
ARXYHDRVLXXDC

SEQ ID NO: 12 - protein, 13 aa, Artificial Sequence
  Synthetic Peptide
  MISC_FEATURE (3)..(3): Xaa is Ahp
  MISC_FEATURE (7)..(7): Xaa is Har
  MISC_FEATURE (10)..(10): Xaa is Bph
  MISC_FEATURE (11)..(11): Xaa is Ahp
ARXYHDXVLXXDC

SEQ ID NO: 13 - protein, 13 aa, Artificial Sequence
  Synthetic Peptide
  MISC_FEATURE (3)..(3): Xaa is Ahp
  MISC_FEATURE (9)..(9): Xaa is MetO2
  MISC_FEATURE (10)..(10): Xaa is Bph
  MISC_FEATURE (11)..(11): Xaa is Ahp
ARXYHDQVXXXDC

SEQ ID NO: 14 - protein, 13 aa, Artificial Sequence
  Synthetic Peptide
  MISC_FEATURE (3)..(3): Xaa is Ahp
  MISC_FEATURE (9)..(9): Xaa is MetO2
  MISC_FEATURE (10)..(10): Xaa is Bph
  MISC_FEATURE (11)..(11): Xaa is Ahp
ARXYHDEVXXXDC

SEQ ID NO: 15 - protein, 13 aa, Artificial Sequence
  Synthetic Peptide
  MISC_FEATURE (3)..(3): Xaa is Ahp
  MISC_FEATURE (9)..(9): Xaa is MetO2
  MISC_FEATURE (10)..(10): Xaa is Bph
  MISC_FEATURE (11)..(11): Xaa is Ahp
ARXYHDRVXXXDC

SEQ ID NO: 16 - protein, 13 aa, Artificial Sequence
  Synthetic Peptide
  MISC_FEATURE (3)..(3): Xaa is Ahp
  MISC_FEATURE (7)..(7): Xaa is Har
  MISC_FEATURE (9)..(9): Xaa is MetO2
  MISC_FEATURE (10)..(10): Xaa is Bph
  MISC_FEATURE (11)..(11): Xaa is Ahp
ARXYHDXVXXXDC

SEQ ID NO: 17 - protein, 13 aa, Artificial Sequence
  Synthetic Peptide
  MISC_FEATURE (1)..(1): Xaa is Lys(MePEG4c)
  MISC_FEATURE (3)..(3): Xaa is Ahp
  MISC_FEATURE (10)..(10): Xaa is Bph
  MISC_FEATURE (11)..(11): Xaa is Ahp
XRXYHDEVLXXDC

SEQ ID NO: 18 - protein, 13 aa, Artificial Sequence
  Synthetic Peptide
  MISC_FEATURE (2)..(2): Xaa is Lys(MePEG4c)
  MISC_FEATURE (3)..(3): Xaa is Ahp
  MISC_FEATURE (10)..(10): Xaa is Bph
  MISC_FEATURE (11)..(11): Xaa is Ahp
AXXYHDEVLXXDC

SEQ ID NO: 19 - protein, 13 aa, Artificial Sequence
  Synthetic Peptide
  MISC_FEATURE (3)..(3): Xaa is Lys(MePEG4c)
  MISC_FEATURE (10)..(10): Xaa is Bph
  MISC_FEATURE (11)..(11): Xaa is Ahp
ARXYHDEVLXXDC

SEQ ID NO: 20 - protein, 13 aa, Artificial Sequence
  Synthetic Peptide
  MISC_FEATURE (1)..(1): Xaa is da
  MISC_FEATURE (3)..(3): Xaa is Ahp
  MISC_FEATURE (10)..(10): Xaa is Bph
  MISC_FEATURE (11)..(11): Xaa is Ahp
XRXYHDEVLXXDC

SEQ ID NO: 21 - protein, 13 aa, Artificial Sequence
  Synthetic Peptide
  MISC_FEATURE (3)..(3): Xaa is Ahp
  MISC_FEATURE (10)..(10): Xaa is Bph
  MISC_FEATURE (11)..(11): Xaa is Ahp
ARXAHDEVLXXDC

SEQ ID NO: 22 - protein, 13 aa, Artificial Sequence
  Synthetic Peptide
  MISC_FEATURE (3)..(3): Xaa is Ahp
  MISC_FEATURE (5)..(5): Xaa is W6N
  MISC_FEATURE (10)..(10): Xaa is Bph
  MISC_FEATURE (11)..(11): Xaa is Ahp
ARXYXDEVLXXDC

SEQ ID NO: 23 - protein, 13 aa, Artificial Sequence
  Synthetic Peptide
  MISC_FEATURE (3)..(3): Xaa is Ahp
  MISC_FEATURE (10)..(10): Xaa is Bph
  MISC_FEATURE (11)..(11): Xaa is Ano
ARXYHDEVLXXDC

SEQ ID NO: 24 - protein, 13 aa, Artificial Sequence
  Synthetic Peptide
  MISC_FEATURE (3)..(3): Xaa is Ahp
  MISC_FEATURE (10)..(10): Xaa is Bph
  MISC_FEATURE (11)..(11): Xaa is Ado
ARXYHDEVLXXDC

SEQ ID NO: 25 - protein, 13 aa, Artificial Sequence
  Synthetic Peptide
  MISC_FEATURE (3)..(3): Xaa is Ahp
  MISC_FEATURE (10)..(10): Xaa is Bph
  MISC_FEATURE (11)..(11): Xaa is PhNle
ARXYHDEVLXXDC

SEQ ID NO: 26 - protein, 13 aa, Artificial Sequence
  Synthetic Peptide
  MISC_FEATURE (3)..(3): Xaa is Ahp
  MISC_FEATURE (10)..(10): Xaa is Bph
  MISC_FEATURE (11)..(11): Xaa is PhNva
ARXYHDEVLXXDC

SEQ ID NO: 27 - protein, 13 aa, Artificial Sequence
  Synthetic Peptide
  MISC_FEATURE (3)..(3): Xaa is Ahp
  MISC_FEATURE (10)..(10): Xaa is Bph
  MISC_FEATURE (11)..(11): Xaa is Ahp
ARXYHDEVLXXAC

SEQ ID NO: 28 - protein, 13 aa, Artificial Sequence
  Synthetic Peptide
  MISC_FEATURE (2)..(2): Xaa is Cit
  MISC_FEATURE (3)..(3): Xaa is Ahp
  MISC_FEATURE (10)..(10): Xaa is Bph
  MISC_FEATURE (11)..(11): Xaa is Ahp
AXXYHDEVLXXDC

SEQ ID NO: 29 - protein, 13 aa, Artificial Sequence
  Synthetic Peptide
  MISC_FEATURE (2)..(2): Xaa is F3G
  MISC_FEATURE (3)..(3): Xaa is Ahp
  MISC_FEATURE (10)..(10): Xaa is Bph
  MISC_FEATURE (11)..(11): Xaa is Ahp
AXXYHDEVLXXDC

SEQ ID NO: 30 - protein, 13 aa, Artificial Sequence
  Synthetic Peptide
  MISC_FEATURE (2)..(2): Xaa is RNMe
  MISC_FEATURE (3)..(3): Xaa is Ahp
  MISC_FEATURE (10)..(10): Xaa is Bph
  MISC_FEATURE (11)..(11): Xaa is Ahp
AXXYHDEVLXXDC

SEQ ID NO: 31 - protein, 13 aa, Artificial Sequence
  Synthetic Peptide
  MISC_FEATURE (2)..(2): Xaa is RNNdMe
  MISC_FEATURE (3)..(3): Xaa is Ahp
  MISC_FEATURE (10)..(10): Xaa is Bph
  MISC_FEATURE (11)..(11): Xaa is Ahp
AXXYHDEVLXXDC

SEQ ID NO: 32 - protein, 13 aa, Artificial Sequence
  Synthetic Peptide
  MISC_FEATURE (2)..(2): Xaa is RNdMe
  MISC_FEATURE (3)..(3): Xaa is Ahp
  MISC_FEATURE (10)..(10): Xaa is Bph
  MISC_FEATURE (11)..(11): Xaa is Ahp
AXXYHDEVLXXDC

SEQ ID NO: 33 - protein, 13 aa, Artificial Sequence
  Synthetic Peptide
  MISC_FEATURE (2)..(2): Xaa is hCit
  MISC_FEATURE (3)..(3): Xaa is Ahp
  MISC_FEATURE (10)..(10): Xaa is Bph
  MISC_FEATURE (11)..(11): Xaa is Ahp
AXXYHDEVLXXDC

SEQ ID NO: 34 - protein, 13 aa, Artificial Sequence
  Synthetic Peptide
  MISC_FEATURE (2)..(2): Xaa is 4Py2NH2
  MISC_FEATURE (3)..(3): Xaa is Ahp
  MISC_FEATURE (10)..(10): Xaa is Bph
  MISC_FEATURE (11)..(11): Xaa is Ahp
AXXYHDEVLXXDC

(19) United States
(12) Patent Application Publication
(10) Pub. No.: US 2025/0263440 A1
(43) Pub. Date: Aug. 21, 2025

(54) CD38-BINDING AGENTS AND USES THEREOF

(71) Applicant: PeptiDream Inc., Kawasaki-shi, Kanagawa (JP)

(72) Inventors: Patrick Crawford REID, Kawasaki-shi, Kanagawa (JP); Masaki OHUCHI, Kawasaki-shi, Kanagawa (JP); Naoko NAKAMURA, Kawasaki-shi, Kanagawa (JP); Hiroko GOTO, Kawasaki-shi, Kanagawa (JP); Takeru EHARA, Kawasaki-shi, Kanagawa (JP); Masatoshi TAKUWA, Kawasaki-shi, Kanagawa (JP)

(73) Assignee: PeptiDream Inc., Kawasaki-shi, Kanagawa (JP)

(21) Appl. No.: 17/624,351

(22) PCT Filed: Jun. 24, 2020
(86) PCT No.: PCT/JP2020/024869

§ 371 Date: Jan. 3, 2022

Related U.S. Application Data
(60) Provisional application No. 62/870,629, filed on Jul. 3, 2019.

(51) Int. Cl. C07K 7/50; A61K 38/00; G01N 33/574

(52) U.S. Cl. CPC C07K 7/50; G01N 33/574; A61K 38/00; G01N 2333/70596

(57) ABSTRACT

A peptide, compound, or conjugate that can bind CD38. The CD38-binding peptide, compound, or conjugate has a sequence selected from the group consisting of SEQ ID NOs. 1-34. The CD38-binding peptides, compounds, or conjugates are useful for treating CD38-associated conditions, disorders, or diseases, such as cancer, leukemia, or myelomas.

### INCORPORATION OF SEQUENCE LISTING

This application contains Sequence Listing in ASCII text file submitted via EFS-Web. The Sequence Listing is incorporated by reference herein. The Sequence Listing text file is named “HR023-SequenceListing,” created on Oct. 18, 2023, having 16 KB in size.

### TECHNICAL FIELD

The present invention relates to CD38-binding peptide agent, and peptides which specifically bind to human CD38.

### BACKGROUND

CD38 is expressed by various types of cells in human and has a number of important functions. In some embodiments, CD38 is associated with various conditions, disorders or diseases.

CD38, also known as cyclic ADP ribose hydrase, is a Type II transmembrane glycoprotein which consists of long C-terminal extracellular domain and short N-terminal intracellular domain.

CD38 is also a member of ‘superfamily’ of membrane-bound or soluble enzyme, ADP-ribosyl cyclase which includes CD157 and Aplysia ADPR. This superfamily enzymes convert NAD to cyclic ADP ribose or nicotinic acid-adenine dinucleotide phosphate.

In addition, CD38 is reported to be involved in CA2+ signal pathway and other signal pathways such as Phospholipase Cy, ZAP-70, syk and c-cbl. These reports suggested that CD38 plays important roles as signaling molecule in the normal development, maturation and activation of Lymphoid cells. Many functions among hematopoietic cells is caused by CD38-mediated signaling pathway, which includes increase of Lymphocyte, release of Cytokinin, development and death of B cell and Myeloid cells and Induction of dendritic cell maturation. Also, CD38 is known to be involved in cell-cell adhesion.

In this way, CD38 is an important molecule involved in many biological phenomena. Recently, it is greatly paid attention that CD38 relates to Malignant tumors, especially hematologic malignancies. It is well known that those malignancies have increased expression of CD38. In addition to the fact, pluripotent stem cells at first stage don't express CD38 (CD38⁻), it is supposed that CD38 is a potential target of antibody therapy for hematologic malignancy and chronic lymphogenous leukemia.

### CITATION LIST

### Patent Literature

PLT1: WO2016/146639

### SUMMARY OF INVENTION

### Technical Problem

It has been realized that cancer therapy using antibody which specifically binds to CD38. There are many documents describing anti-CD38 antibody (exam. Patent 1, 2). Patent 2 discloses antibody dependent cellular cytotoxicity and CDC Complement-Dependent Cytotoxicity activity. Further, Patent 3-6 reported that usage of anti-CD38 antibody in combination with cell toxic chemical compounds such as Cytarabine, Vincristinem Cyclo phosmide and Melphalan.

CD38 is also known as bio marker molecule to determine HIV infection, leukemia, myeloma, solid tumor, type diabetes, bone metabolism and other diseases or status genetically determined. Especially, it is well known to use CD38 as a prognostic marker of leukemia (Ibrahim, S. et al. (2001) CD38 expression as an important prognostic factor in B-cell chronic lymphocytic leukemia. Blood 98: 181-186) or marker of multiple myeloma.

However, antibody sometimes cannot reach the target due to its large size. Because this problem will be a bog problem for therapies using conjugate of antibody and cytotoxic compounds, molecules which are smaller than antibody but especially bind to CD38 have been sought.

### Solution to Problem

Among other things, the present disclosure provides technologies (e.g., compounds, compositions, methods, etc.) for modulating CD38 activities. In some embodiments, the present disclosure provides technologies that can bind to CD38, and can modulate one or more properties and/or functions of CD38 and/or entities that comprise and/or expresses CD38.

Particularly, in some embodiments, the present disclosure provides compounds that can bind to CD38 with high affinity. In some embodiments, a provided compound is or comprises a peptide moiety comprising one or more residues of amino acids or analogs thereof. In some embodiments, a provided compound is or comprises (Xaa)y or a salt form thereof as described herein. In some embodiments, a provided compound is or comprises -Xaa^{T1}-Xaa^{T2}-(Xaa)y′-Xaa^{T3}-Xaa^{T4}-Xaa^{T5}- or a salt form thereof as described herein. In some embodiments, a provided compound is or comprises -Xaa^{T6}-(Xaa)y′-Xaa^{T7}-Xaa^{T8}-Xaa^{T9}-Xaa^{T10}-Xaa^{T11}- or a salt form thereof as described herein.

In some embodiments, provided compounds are much smaller than CD38 antibodies in size and/or can provide a number of benefits compared to CD38 antibodies for various uses, e.g., as detection, diagnosis, or therapeutic agents or fragments thereof. As those skilled in the art will appreciate, in some embodiments, provided compounds are much easier to manufacture compared to antibodies, particularly at large-scale for commercial and/or therapeutic purposes. In some embodiments, provided compounds can be manufactured synthetically, therefore providing high purity and/or uniformity compared to antibodies. In some embodiments, provided compounds and compositions thereof provide significantly improved stability, bioavailability, delivery properties and other benefits for various uses including as therapeutic agents or fragments thereof.

In some embodiments, provided compounds are conjugates, comprising a first moiety, e.g., peptide moieties as described herein, that bind to CD38, a second moiety that is useful for various purposes, e.g., detection, diagnostic, therapeutic, etc., and optionally a linker moiety linking the first and the second moieties. Various technologies for preparing such conjugates, including chemistry, useful second moieties and linker moieties, etc. are available in the art and can be utilized in accordance with the present disclosure. For example, in some embodiments, a provided compound is a peptide-drug conjugate (PDC), wherein it comprises a peptide moiety that binds to CD38, a drug moiety corresponding to a drug, and optionally a linker linking the peptide moiety and the drug moiety. In some embodiments, PDCs utilize CD38-binding moieties to target specific targets, e.g., diseased cells expressing CD38, and specifically deliver drug moieties (in other types of conjugates, a second conjugate can be detection or diagnostic moieties, depending on the desired uses). In some embodiments, a drug is a toxin. In some embodiments, a drug is a small molecule inhibitor, e.g., of various therapeutically relevant enzymes or other types of proteins. In some embodiments, a drug is a nucleic acid drug. Those skilled in the art will appreciate that, among other things, many technologies in the field of antibody-drug conjugates (ADCs) can be utilized in accordance with the present disclosure. In some embodiments, a PDC is provided by, for example, replacing an antibody moiety of an ADC with a CD38-binding moiety as described herein, e.g., one of various cyclic peptide moieties as described herein, with optional optimization of the drug and/or linker moieties.

In some embodiments, the present disclosure provides methods for identifying, assessing, preparing and using provided compounds and compositions, e.g., those described in the examples.

### Advantageous Effects of Invention

The present disclosure provides technologies (e.g., compounds, compositions, methods, etc.) for modulating CD38 activities.

### BRIEF DESCRIPTION OF DRAWINGS

FIG. 1. Exemplary SPR results of compound I-1.

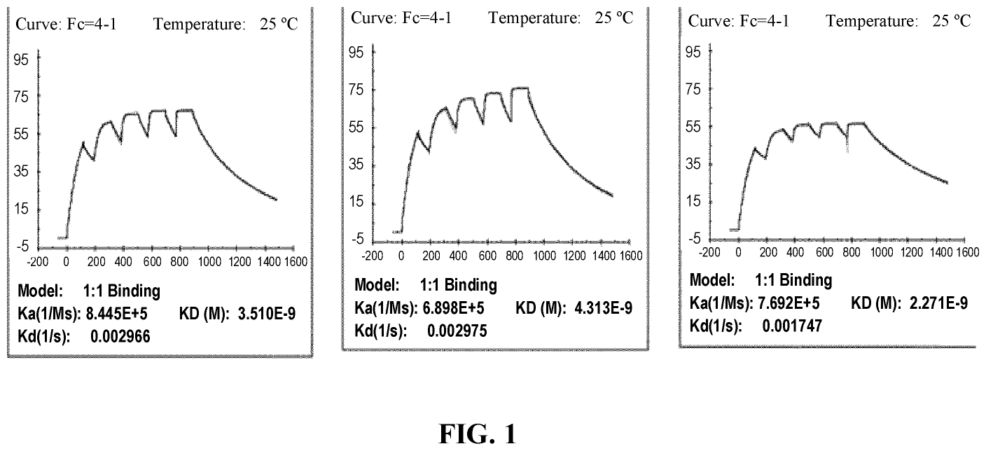

FIG. 2. Exemplary SPR results of compound I-2.

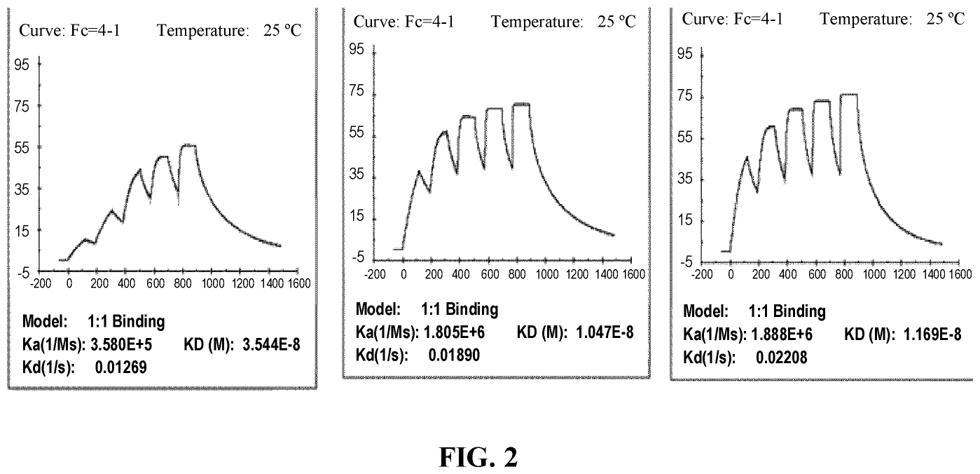

### DETAILED DESCRIPTION OF THE INVENTION

### 1. General Description of Certain Embodiments

Among other things, the present disclosure provides agents, e.g., various compounds illustrated herein, that can bind to CD38, thereby modulating CD38 functions and/or delivery useful agents to targets comprising and/or expressing CD38. In some embodiments, a provided compound is a peptide and/or comprises peptide moieties, in many instances, cyclic peptide moieties, that can bind to CD38. In some embodiments, a peptide moiety comprises one or more residues of amino acids or amino acid analogs.

In some embodiments, an amino acid analog is a compound in which the amino group and/or carboxylic acid group are independently replaced with an optionally substituted aliphatic or heteroaliphatic moiety. As those skilled in the art will appreciate, many amino acid analogs, which mimics structures, properties and/or functions of amino acids, are described in the art and can be utilized in accordance with the present disclosure.

The present invention provides to CD38-binding peptides and therapy, diagnostic and determine method of CD38 using regent containing the peptides thereof.

The first aspect of the invention relates to a CD38-binding peptide, a pharmaceutically acceptable salt thereof or a pharmaceutically acceptable solvate thereof, wherein the CD38-binding peptide is
(1) a polypeptide having an amino acid sequence represented by SEQ ID NO. 1 or 2:

(SEQ ID NO. 1)
Ala Arg Ahp Tyr His Asp Gly Val Leu Bph Ahp Asp
Cys,
(SEQ ID NO. 2)
Ala Leu His MePhe Val Leu Pro Bph Val Trp Val Cys;

(2) a polypeptide having an amino acid sequence represented by SEQ ID NO. 1 or 2 wherein the Ala at the N-terminal is a chloroacetylated Ala;
(3) a polypeptide having an amino acid sequence with deletions, additions, substitutions or insertion of one or more amino acids in SEQ ID NO. 1 or 2, which does not comprises an amino acid sequence with deletion of Cys at the C terminal in SEQ ID NO. 1 or 2;
(4) a polypeptide having an amino acid sequence represented by SEQ ID NO. 1 or 2 wherein the Ala at the N-terminal is a chloroacetylated Ala with deletions, additions, substitutions or insertion of one or more amino acids in SEQ ID NO. 1 or 2, which does not comprises an amino acid sequence with deletion of Cys at the C terminal in SEQ ID NO. 1 or 2; or
(5) a polypeptide in accordance with one of the above (1) to (4) wherein the polypeptide has a cyclized structure.

The preferred embodiment of the invention is that the CD38-binding peptide has an amino acid sequence represented by one of SEQ IDs NO. 1 to 34.

The preferred embodiment of the invention is that the CD38-binding peptide has a cyclized structure.

The second aspect of the invention relates to a chemical compound comprising the above CD38-binding peptide, a pharmaceutically acceptable salt thereof or a pharmaceutically acceptable solvate thereof, in which the CD38-binding peptide, a pharmaceutically acceptable salt thereof or a pharmaceutically acceptable solvate further comprises a linker moiety.

The preferred embodiment of the invention is that the linker moiety comprises polyethylene glycol, PEG.

The third aspect of the invention relates to a pharmaceutical composition for treating CD38-associated conditions, disorders or diseases comprising: the above CD38-binding peptide, a pharmaceutically acceptable salt thereof or a pharmaceutically acceptable solvate thereof, and a pharmaceutically acceptable carrier.

The preferred embodiment of the invention is that the CD38-associated conditions, disorders or diseases is cancer.

The preferred embodiment of the invention is that the CD38-associated conditions, disorders or diseases is leukemia or myelomas.

The preferred embodiment of the invention is that the CD38-associated conditions, disorders or diseases is B-cell non-Hodgkin's Lymphoma (NHL), multiple myeloma (MN), acute myeloid leukaemia (AML), acute lymphoblastic leukaemia (B-cell ALL) or chronic lymphocytic leukaemia (CLL). As disclosed in WO-2017-025323 pamphlet, example of the CD38-associated conditions are NHL, MM, AML, B-cell ALL and CLL.

The preferred embodiment of the invention is that the CD38-associated conditions, disorders or diseases is multiple myeloma (MN).

The forth aspect of the invention relates to an agent for detecting cancer, wherein the agent comprises the above CD38-binding peptide, a pharmaceutically acceptable salt thereof or a pharmaceutically acceptable solvate thereof.

### 2. Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included. Unless otherwise specified, compounds described herein may be provided and/or utilized in a salt form, particularly a pharmaceutically acceptable salt form.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups. In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference.

Subject: As used herein, the term “subject” refers to any organism to which a compound or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a compound described herein.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated,” as used herein, means that a moiety has one or more units of unsaturation.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds are within the scope of the present disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of the present disclosure. Such compounds are useful, for example, as analytical tools, as probes in biological assays, or as therapeutic agents in accordance with the present disclosure.

### 3. Description of Exemplary Embodiments

Amino acids: In the present specification, amino acid means any known proteinogenic amino acid which are naturally encoded or found in the genetic code of any organism, or non non-proteinogenic amino acid which are not naturally encoded or found in the genetic code of any organism. Examples of non-proteinogenic amino acids include α,α-disubstituted amino acids (α-methylalanine etc.), N-alkyl-α-amino acids, N-alkyl-α-D-amino acids, whose main chain structure is different from the natural type. Examples thereof include, but are not limited to, β-amino acids and amino acids having a side chain structure different from that of the natural type (such as norleucine, homohistidine, and hydroxyproline).

CD38: CD38 (cluster of differentiation 38, also known as cyclic ADP ribose hydrolase) is expressed by various types of cells and performs a number of functions. It has been reported that CD38 was found on the surface of many immune cells, e.g., CD4⁺, CD8⁺, B lymphocytes and natural killer cells, etc., as a glycoprotein. Other functions, e.g., cell adhesion, signal transduction and calcium signaling were also reported for CD38. A preferred CD38 is a human CD38.

CD38 is associated with various conditions, disorders or diseases, e.g., HIV infection, leukemia, myelomas, solid tumors, CLL, MM, APL (described below) etc.

Daratumumab, an antibody which targets CD38, has been approved in treating multiple myeloma.

CD38 is also a non-lineage-restricted, type II transmembrane glycoprotein that synthesizes and hydrolyzes cyclic adenosine 5′-diphosphate-ribose, an intracellular calcium ion mobilizing messenger. The release of soluble protein and the ability of membrane-bound protein to become internalized indicate both extracellular and intracellular functions for the protein. This protein has an N-terminal cytoplasmic tail, a single membrane-spanning domain, and a C-terminal extracellular region with four N-glycosylation sites. Crystal structure analysis demonstrates that the functional molecule is a dimer, with the central portion containing the catalytic site. It is used as a prognostic marker for patients with chronic lymphocytic leukemia. Alternative splicing results in multiple transcript variants.

CD38-Binding Agents: In some embodiments, a present disclosure agent can bind to CD38. In this description, a peptide specifically binds to CD38 (also referred as CD38-binding peptide) means any peptide which is able to bind especially to CD38. It can be confirmed by a person skilled in the art according to a well-known method.

The agent may be a CD38-binding peptide, a pharmaceutically acceptable salt thereof or a pharmaceutically acceptable solvate thereof, in which the CD38-binding peptide is:
(1) a polypeptide having an amino acid sequence represented by SEQ ID NO. 1 or 2: Ala Arg Ahp Tyr His Asp Gly Val Leu Bph Ahp Asp Cys (SEQ ID NO.1), Ala Leu His MePhe Val Leu Pro Bph Val Trp Val Cys (SEQ ID NO.2);
(2) a polypeptide having an amino acid sequence represented by SEQ ID NO. 1 or 2 wherein the Ala at the N-terminal is a chloroacetylated Ala;
(3) a polypeptide having an amino acid sequence with deletions, additions, substitutions or insertion of one or more amino acids in SEQ ID NO. 1 or 2, which does not comprises an amino acid sequence with deletion of Cys at the C terminal in SEQ ID NO. 1 or 2;
(4) a polypeptide having an amino acid sequence represented by SEQ ID NO. 1 or 2 wherein the Ala at the N-terminal is a chloroacetylated Ala with deletions, additions, substitutions or insertion of one or more amino acids in SEQ ID NO. 1 or 2, which does not comprises an amino acid sequence with deletion of Cys at the C terminal in SEQ ID NO. 1 or 2; or
(5) a polypeptide in accordance with one of the above (1) to (4) wherein the polypeptide has a cyclized structure.

CD38 related diseases: It is reported that CD38 are expressed on the immune system cells such as T cells or B cells of healthy person. In case of disease, it is determined to increase of CD38 expression or express specifically on the cells which normally no expression is determined. Some diseases are known to have relationship with CD38; for example, tumor, especially cancer or leukemia. For further details;

Chronic lymphocytic leukemia (CLL): CLL is an ordinary adults leukemia that is caused by the accumulation of small B lymphocytes (CD19+/CD5+/CD23+) in blood, blood marrow, Lymph node and other lymphoid tissues. It is possible to determine the progression of disease stage by detection of increased expression of CD38.

Multiple myeloma (MM): MM is a malignant tumor characterized by the accumulation of monoclonal plasm cells, high concentrated monoclonal immunoglobulin (Ig) in blood plasma, urine, solubility bone mutation.

Acute promyelocytic leukemia (APL): APL is a unique subtype of acute leukemia which is characterized by inhibition of white blood cell differentiation during its promyelocyte stage. Over expression of CD38 on granulocyte is observed in patients of this disease.

Other diseases: It is reported that CD38 has relation with non-Hodgkin lymphoma, B and T cell acute lymphotic leukemia, Acute myeloid leukemia, Hodgkin lymphoma, and chronic myeloid leukemia.

Deletions, additions, substitutions or insertion of amino acids: Amino acid substitution means substitution with a known amino acid, preferably a conservative amino acid substitution. Typically, substitution of conservative amino acid residue would not affect the mechanism of the protein or peptides. Examples of amino acid groups having side chains with similar chemical properties include 1) aliphatic side chains: glycine, alanine, valine, leucine, and isoleucine; 2) aliphatic hydroxyl side chains: serine and threonine; 3). Amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) Sulfur-containing side chains: Includes cysteine and methionine. Conservative amino acid substitutions are: valine-leucine-isoleucine, phenylalanine-tyrosine-tryptophan, lysine-arginine, alanine-valine, glutamic acid-aspartic acid, and asparagine-glutamine. Aforementioned amino acids can be proteinic or non-proteinic amino acids. Throughout the specification, the term “one or more amino acids” refers to an amino acid or amino acids that may be deleted, substituted, inserted, and/or added. In addition, the term “one or more amino acids” in this specification may refer to one or several amino acids in some cases. This present invention includes, but not limited to, a peptide composed of an amino acid sequence having deletion, substitution, insertion, and/or addition of 1 to 5 amino acids, preferably 4 or less, 3 or less, 2 or less, more preferably one amino acid or less in an amino acid sequence represented by one of SEQ IDs NO. 1 to 34 and having activity to bind to CD38.

Surgery or therapy and diagnostic of CD38 related diseases: This invention can surgery the CD38 related diseases by binding CD38. For example, it enables to specifically kill or injury cell expression CD38, directly or indirectly, especially cancer cells using conjugate of CD38-binding peptides of this invention and cell toxic chemical compound. Simultaneously, it becomes possible to diagnose the expression profiles of CD38 in vivo by detecting the marker which is conjugated to the CD38-binding peptide and administrated to a patient. For example, it is possible to diagnose the progression of disease by administration and detecting the amount of CD38-binding peptide in a CLL patient.

Target: In some embodiments, the present disclosure provides technologies for selectively directing agents comprising CD38-binding moieties to desired target sites comprising one or more targets. As those skilled in the art will appreciate, provided technologies are useful for various types of targets, particularly those comprising CD38.

In some embodiments, targets are damaged or defective tissues. In some embodiments, a target is a damaged tissue. In some embodiments, a target is a defective tissue. In some embodiments, a target is associated with a disease, disorder or condition, e.g., cancer, wound, etc. In some embodiments, a target is a tumor. In some embodiments, targets are or comprise diseased cells. In some embodiments, targets are or comprise cancer cells. In some embodiments, a target is a foreign object. In some embodiments, a target is or comprises an infectious agent. In some embodiments, a target is a microbe. In some embodiments, a target is or comprises bacteria. In some embodiments, a target is or comprises viruses. In some embodiments, targets comprise or express CD38.

In many embodiments, targets are tissues and/or cells associated with diseases, disorders or conditions, particularly various types of cancers. In some embodiments, targets are or comprise cells associated with conditions, disorders or diseases. In some embodiments, targets are or comprise cells associated with cancer. In some embodiments, cells comprise or express CD38.

Conjugates: In some embodiments, the present disclosure provides agents that are conjugate compounds. In some embodiments, a provided conjugate comprises a first moiety which can bind to CD38, and a second moiety, which can be a variety of structures for a number of purposes, e.g., a moiety of a detection agent, a diagnostic agent, or a therapeutic, etc, and optionally a linker which links the first moiety and a second moiety.

In some embodiments, CD38-binding peptide and materials which is desired to be delivered can be connected. Materials desired to be delivered is not limited to, for example, materials can be the material used for the therapy of CD38 related diseases. For example, it could be anti-CD38 antibody, cell-toxic chemical compound or radio-isotope labeled chemical materials. It also could be materials that directly injury the cells bound to CD38-binding peptides, or materials that indirectly injury those cells, for example, anti-cancer materials (such as Daratumumab) that affect on cancer cells by binding CD38 antigen expressed on the surface of cancer cells of hematopoietic tumor including multiple myeloma via Complement-Dependent Cytotoxicity (CDC), or antibody-dependent cellular cytotoxicity (ADCP).

In addition, such materials could be any tags useful for diagnosing CD38-related diseases. For example, antibody with directable labels, bio stimulated tag, PET regent. For clarity, one or more amino acids of the infection can be directly or indirectly labeled when using for diagnosing.

In some embodiments, a CD38 binding moiety, e.g., a first moiety, is or comprises -PM, wherein H-PM is a compound of Table 1 or a salt form thereof. In some embodiments, H-PM is I-1 or a salt thereof. In some embodiments, H-PM is I-2 or a salt thereof. In some embodiments, a first moiety, e.g., -PM, is or comprises:

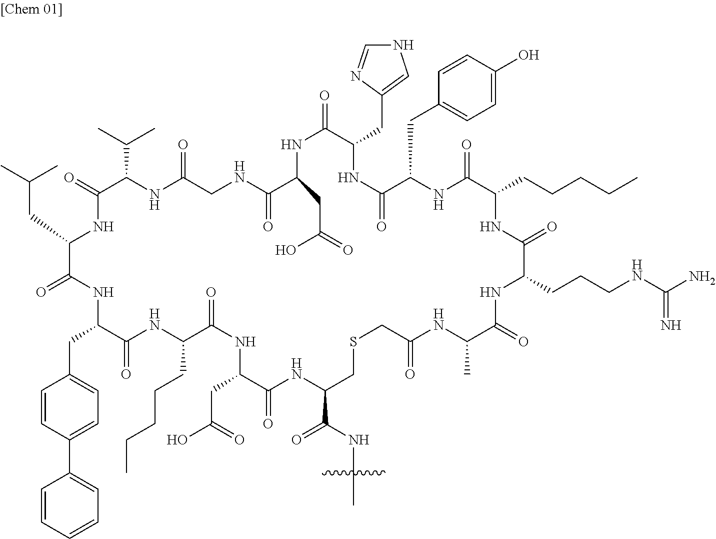 [SMILES: *NC(=O)[C@@H]1CSCC(=O)N[C@@H](C)C(=O)N[C@@H](CCCNC(=N)N)C(=O)N[C@@H](CCCCC)C(=O)N[C@@H](Cc2ccc(O)cc2)C(=O)N[C@@H](Cc2c[nH]cn2)C(=O)N[C@@H](CC(=O)O)C(=O)NCC(=O)N[C@@H](C(C)C)C(=O)N[C@@H](CC(C)C)C(=O)N[C@@H](Cc2ccc(-c3ccccc3)cc2)C(=O)N[C@@H](CCCCC)C(=O)N[C@@H](CC(=O)O)C(=O)N1]

or a salt form thereof.

In some embodiments, a first moiety, e.g., -PM, is or comprises

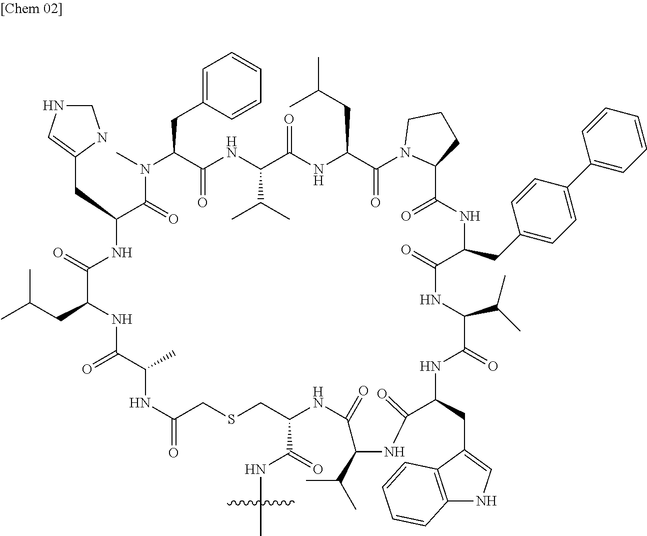 [SMILES: *NC(=O)[C@@H]1CSCC(=O)N[C@@H](C)C(=O)N[C@@H](CC(C)C)C(=O)N[C@@H](CC2=CNCN2)C(=O)N(C)[C@@H](Cc2ccccc2)C(=O)N[C@@H](C(C)C)C(=O)N[C@@H](CC(C)C)C(=O)N2CCC[C@H]2C(=O)N[C@@H](Cc2ccc(-c3ccccc3)cc2)C(=O)N[C@@H](C(C)C)C(=O)N[C@@H](Cc2c[nH]c3ccccc23)C(=O)N[C@@H](C(C)C)C(=O)N1]

or a salt form thereof.

As appreciated by those skilled in the art, provided conjugates can comprise moieties of a variety of useful agents (e.g., as second moieties) for a number of purposes. Suitable agents/moieties for detection, diagnostic, therapy, etc. are widely known in the art and can be utilized in accordance with the present disclosure.

For example, in some embodiments, useful agents are therapeutics. In some embodiments, useful agents are FDA-approval therapeutics, or those that were or are in clinical trials. In some embodiments, useful agents are cancer therapeutics. In some embodiments, useful agents kill cells or inhibit cell growth. In some embodiments, useful agents are toxins, e.g., those can inhibit cell growth and/or kill cells. In some embodiments, useful agents are radiopharmaceuticals.

In some embodiments, a second agent is selected from a small molecule, a nucleic acid, a polypeptide, a protein, a carbohydrate, a lipid and any combinations thereof. In some embodiments, a second moiety is a moiety of such a second agent.

In some embodiments, a second moiety is a payload of an antibody-drug conjugates, which have been extensively described in the art.

In some embodiments, a second moiety is useful for detection and/or diagnosis, such as a moiety that can provide a signal which can be detected under suitable conditions using suitable technologies. For example, in some embodiments, a second moiety is or comprises a fluorescent moiety. In some embodiments, a second moiety is or comprises a radiolabel (e.g., an isotope for detection). In some embodiments, a second moiety is or comprises a moiety useful for imaging, e.g., PET. In some embodiments, a second moiety is or comprises a RI tag. In some embodiments, a second moiety is or comprises a RI imaging tag. In some embodiments, a second moiety is or comprises a FDG tag. In some embodiments, a second moiety is or comprises a paramagnetic ion tags. In some embodiments, a second moiety is or comprise a solid phase moiety. In some embodiments, a second moiety is or comprise a metal nano tag such as Au or Ag tag. In some embodiments, a second moiety is or comprises an affinity tag, e.g., His tag(poly-His, hexa-His) tag, HA tag, myc tag, FLAG tag, V5 tag S tag, E tag, T7 tag, VSV-G tag, Gly-Glu tag, HSV tag, Strep(II) tag, CBD tag, CBP tag, GST (Glutathione S-transferase) tag, MBP (Maltose Binding Protein) tag, Thioredoxin tag, Biotin Carboxyl Carrier Protein (BCCP) tag, etc. In some embodiments, a second moiety is or comprise a metal a fluorescent tags such as GFP tag, RFP tag, FITC tag, etc. In some embodiments, a second moiety is or comprises a hapten tag. In some embodiments, a second moiety is or comprises a label such as a FLAG tag, HA tag, etc. In some embodiments, a second moiety is or comprises a nucleic acid tag.

Certain useful moieties are described in WO2016/146639, WO2009/140408, WO2015/095895, WO2016/168817, WO2009/092732, Mol. BioSyst., 2016, 12, 1731-1745, Trends Biotechnol. 2012 January; 30(1): 8-16.

A conjugate agent can contain one or more first moieties (e.g., CD38-binding moieties) which can be the same or different, and one or more second moieties which can be the same or different and be for the same or different uses.

In some embodiments, the present disclosure provides methods for preventing or treating a CD38-associated conditions, disorders or diseases, comprising administering to a subject susceptible to or suffering therefrom an effective amount of a provided compound or a pharmaceutically acceptable salt thereof. In some embodiments, the present disclosure provides methods for detecting in a system a target comprising CD38 (e.g., a cell expressing CD38), comprising administering to the system a provided compound or a salt thereof. In some embodiments, the present disclosure provides methods for detecting in a system a target comprising CD38 (e.g., a cell expressing CD38), comprising contacting the target with a provided compound or a salt thereof. In some embodiments, the present disclosure provides a method for diagnosis of a condition, disorder or disease, comprising administering to a subject a provided compound or a salt thereof. In some embodiments, a provided compound is a compound of formula which is selected from I-1 to I-38. In some embodiments, a provided compound is a conjugate. In some embodiments, a provided compound is a conjugate of a compound of formula I-1 to I-38.

Various conjugation chemistry are widely known and practiced in the art and may be utilized to prepare provided conjugates in accordance with the present disclosure. For example, in some embodiments, a first moiety is linked to a first reactive group, optionally through a linker, and a second moiety is linked to a second reactive group, optionally through a linker, and the first reactive group can react with the second reactive group. In some embodiments, reactive groups are or comprise amino groups, acid groups, reactive groups for cycloaddition reactions (e.g., Diels-Alder and variants thereof, Staudinger ligation and variant thereof, click chemistry and variants thereof, etc.) and activated forms thereof. In some embodiments, a first reactive group is an amino group. In some embodiments, a second reactive group is an activated acid.

### Linker Moieties

CD38-binding peptides and martials needed to be delivered to CD38 could be connected with linkers.

Linker (also as cross linkers) can be any publicly known linkers or described herein. In some embodiments, these linkers would be chemical linkers, lipid linkers, peptide (polypeptide) linkers, for example. Or, it would be the combination of chemical linker and peptide linker. For example, these linkers would be a linker that would be disassociated or separated, or stable under specific conditions. Probably, PEG (Polyethylene glycol) linkers or peptide linkers. For example, it can be a PEG consists of 1-24 Ethylene glycol units. Peptide linkers can consist of at least one amino acid. In some embodiments, linker can be an amino acid (for example, it could be any amino acid such as Gly, Lys, Glu, Ser or Ala). In some embodiments, linker can be the combination of PEG linker and peptide linker which is combined with chemical compounds.

Exemplary compounds include those set forth in Table 1, below.

TABLE 1
Exemplary compounds
[Chem 03]

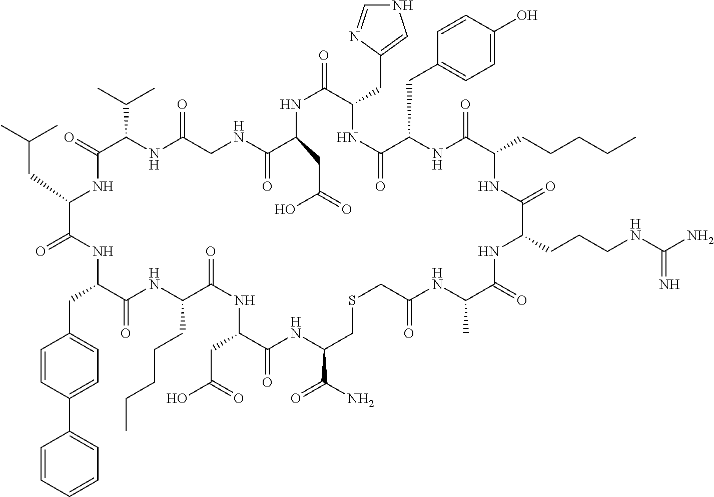 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)CNC(=O)[C@H](CC(=O)O)NC(=O)[C@H](Cc2c[nH]cn2)NC(=O)[C@H](Cc2ccc(O)cc2)NC1=O]

I-1
[Chem 04]

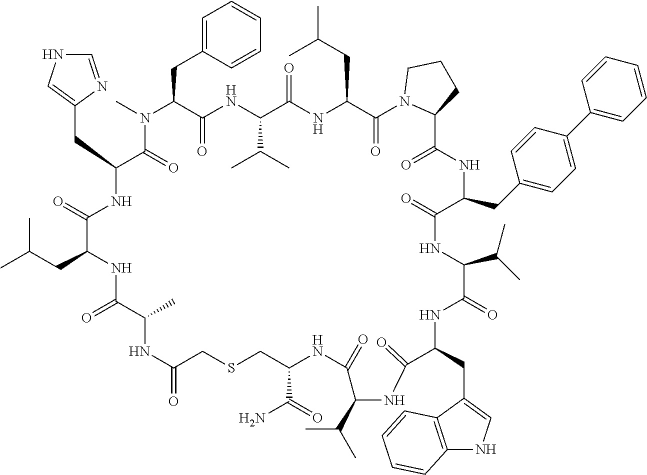 [SMILES: CC(C)C[C@@H]1NC(=O)[C@H](C)NC(=O)CSC[C@@H](C(N)=O)NC(=O)[C@H](C(C)C)NC(=O)[C@H](Cc2c[nH]c3ccccc23)NC(=O)[C@H](C(C)C)NC(=O)[C@H](Cc2ccc(-c3ccccc3)cc2)NC(=O)[C@@H]2CCCN2C(=O)[C@H](CC(C)C)NC(=O)[C@H](C(C)C)NC(=O)[C@H](Cc2ccccc2)N(C)C(=O)[C@H](Cc2c[nH]cn2)NC1=O]

I-2
[Chem 05]

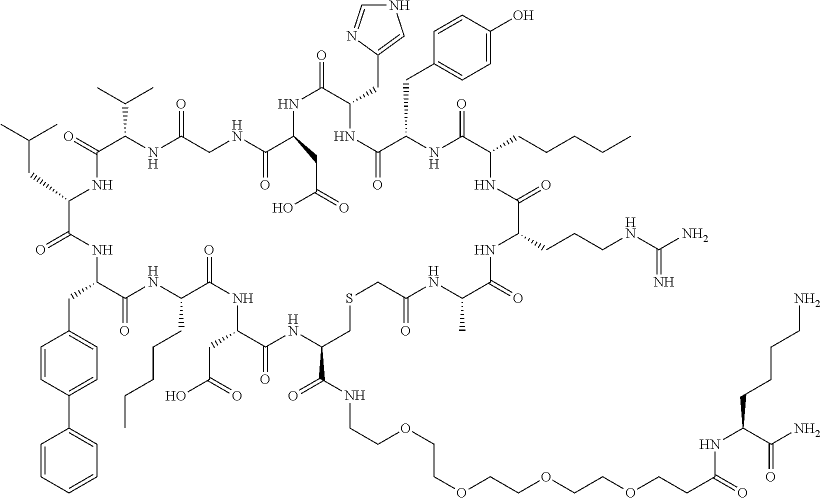 [SMILES: CCCCC[C@@H]1NC(=O)[C@H](CCCNC(=N)N)NC(=O)[C@H](C)NC(=O)CSC[C@@H](C(=O)NCCOCCOCCOCCOCCC(=O)N[C@@H](CCCCN)C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)CNC(=O)[C@H](CC(=O)O)NC(=O)[C@H](Cc2c[nH]cn2)NC(=O)[C@H](Cc2ccc(O)cc2)NC1=O]

I-3
[Chem 06]

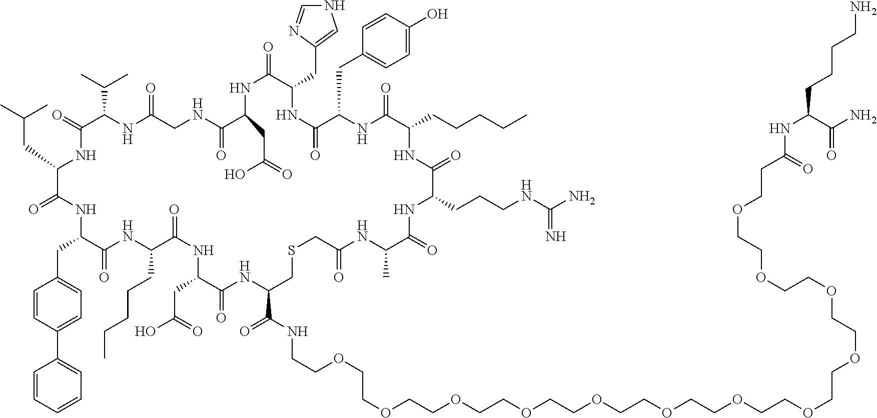 [SMILES: CCCCC[C@@H]1NC(=O)[C@H](CCCNC(=N)N)NC(=O)[C@H](C)NC(=O)CSC[C@@H](C(=O)NCCOCCOCCOCCOCCOCCOCCOCCOCCOCCOCCOCCOCCC(=O)N[C@@H](CCCCN)C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)CNC(=O)[C@H](CC(=O)O)NC(=O)[C@H](Cc2c[nH]cn2)NC(=O)[C@H](Cc2ccc(O)cc2)NC1=O]

I-4
[Chem 07]

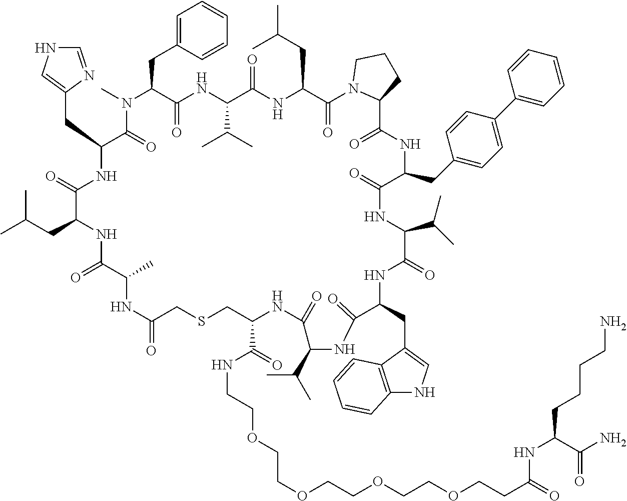 [SMILES: CC(C)C[C@@H]1NC(=O)[C@H](C)NC(=O)CSC[C@@H](C(=O)NCCOCCOCCOCCOCCC(=O)N[C@@H](CCCCN)C(N)=O)NC(=O)[C@H](C(C)C)NC(=O)[C@H](Cc2c[nH]c3ccccc23)NC(=O)[C@H](C(C)C)NC(=O)[C@H](Cc2ccc(-c3ccccc3)cc2)NC(=O)[C@@H]2CCCN2C(=O)[C@H](CC(C)C)NC(=O)[C@H](C(C)C)NC(=O)[C@H](Cc2ccccc2)N(C)C(=O)[C@H](Cc2c[nH]cn2)NC1=O]

I-5
[Chem 08]

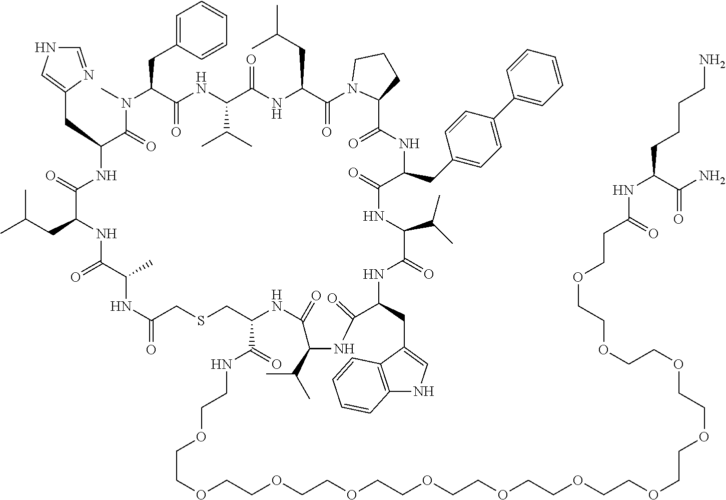 [SMILES: CC(C)C[C@@H]1NC(=O)[C@H](C)NC(=O)CSC[C@@H](C(=O)NCCOCCOCCOCCOCCOCCOCCOCCOCCOCCOCCOCCOCCC(=O)N[C@@H](CCCCN)C(N)=O)NC(=O)[C@H](C(C)C)NC(=O)[C@H](Cc2c[nH]c3ccccc23)NC(=O)[C@H](C(C)C)NC(=O)[C@H](Cc2ccc(-c3ccccc3)cc2)NC(=O)[C@@H]2CCCN2C(=O)[C@H](CC(C)C)NC(=O)[C@H](C(C)C)NC(=O)[C@H](Cc2ccccc2)N(C)C(=O)[C@H](Cc2c[nH]cn2)NC1=O]

I-6

In some embodiments, the present disclosure provides a compound set forth in Table 1, above, or a pharmaceutically acceptable salt thereof.

### 4. General Methods of Providing the Present Compounds

Compounds of the present disclosure may be prepared or isolated in general by synthetic and/or semi-synthetic methods known to those skilled in the art for analogous compounds and by methods described in detail in the Examples, herein.

In some embodiments, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

In some embodiments, leaving groups include but are not limited to, halogens (e.g. fluoride, chloride, bromide, iodide), sulfonates (e.g., —S(O)₂R, mesylate, tosylate, benzenesulfonate, brosylate, nosylate, triflate), diazonium, and the like.

In some embodiments, an oxygen protecting group includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

One of skill in the art will appreciate that compounds of formula I or II may contain one or more stereocenters, and may be present as a racemic or diastereomeric mixture. One of skill in the art will also appreciate that there are many methods known in the art for the separation of isomers to obtain stereoenriched or stereopure isomers of those compounds, including but not limited to HPLC, chiral HPLC, fractional crystallization of diastereomeric salts, kinetic enzymatic resolution (e.g. by fungal-, bacterial-, or animal-derived lipases or esterases), and formation of covalent diastereomeric derivatives using an enantioenriched reagent.

One of skill in the art will appreciate that various functional groups present in compounds of the present disclosure such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the present disclosure are described below in the Exemplification.

Many compounds provided herein comprise peptide moieties. Preparation of such compounds, either through systems comprising biological peptide synthesis machinery (e.g., in vivo or in vitro translation systems) or through chemical synthesis, can be achieved using a variety of technologies. In some embodiments, peptides are prepared through in vitro translation. In some embodiments, peptides are chemically synthesized, e.g., through Fmoc chemistry. Useful technologies for Fmoc synthesis of peptides, including amino acid reagents, protecting groups, coupling conditions, activating reagents, de-protection conditions, purification methods, etc., are widely known in the art and can be readily utilized to prepare provided compounds.

In some embodiments, provided compounds comprise a cyclic peptide moiety. Many suitable cyclization technologies can be utilized in accordance with the present disclosure. For example, in some embodiments, one amino acid, e.g., a N-terminus amino acid, comprises a leaving group (-LG, e.g., —Cl, —Br, —I, etc.), and can react with another amino acid comprising a nucleophile (e.g., —SH of a cysteine side chain). In some embodiments, as illustrated herein, a leaving group is incorporated in an amino acid through —C(O)CH₂-LG, which is bonded to an amino group (e.g., of a N-terminal amino acid residue). In some embodiments, -LG is —Cl. In some embodiments, after deprotection of a peptide cyclization can be achieved under a suitable condition to provide a compound comprising a cyclic peptide moiety. In some embodiments, a provided compound is of formula R^{CN}-(Xaa)y-R^{CS} or a salt thereof, wherein R^{CN} is as described herein, (Xaa)y is as described herein, R^{CS} is R^{CC} or a support, wherein each side chain of Xaa is independently and optionally protected. In some embodiments, R^{CN} comprises a -LG. In some embodiments, R^{CN} is —C(O)-LG. In some embodiments, R^{CN} is —C(O)—Cl. In some embodiments, R^{CN} is bonded to the N-terminal amino group. In some embodiments, R^{CS} is a support, e.g., one for peptide synthesis. In some embodiments, R^{CS} is a resin suitable for peptide synthesis using Fmoc chemistry. In some embodiments, the C-terminus residue is a Cys residue, optionally protected.

As appreciated by those skilled in the art, many technologies are available for preparing conjugates, including those utilizing the reactivity of amino groups, activated acid groups (e.g., NHS esters), nucleophiles, electrophiles, cycloaddition reaction substrates, etc. In some embodiments, a reaction is a biocompatible reaction, which is widely known and practiced in the art and can be utilized in accordance with the present disclosure.

Among other things, the present disclosure provides compounds of high purity. In some embodiments, provided compounds have a purity of 80-100%, e.g., at least 80%, 85%, 90%, 91%, 92%, 93%, 94%, 95%, 96%, 97%, 98% or 99%.

### 5. Technologies for Assessing Provided Compounds

Various assays can be utilized to assess properties and functions of provided compounds. For example, various technologies, e.g., SPR, ELISA, display, FACS, etc., are useful for measuring binding of provided compounds to CD38. In some embodiments, binding is assessed in cell-based assay. A number of methods, both in vitro and in vivo, can be utilized to measure biological impacts of provided compounds and compositions. In some embodiments, Kd for CD38 binding by an agent, e.g., as measured by an assay described herein, is no more than 1000, 500, 200, 150, 120, 100, 90, 80, 70, 60, 50, 40, 30, 20, 10, 9, 8, 7, 6, 5, 4, 3, 2 or 1 nM. In some embodiments, a Kd is no more than 500 nM. In some embodiments, a Kd is no more than 200 nM. In some embodiments, a Kd is no more than 100 nM. In some embodiments, a Kd is no more than 50 nM. In some embodiments, a Kd is no more than 20 nM. In some embodiments, a Kd is no more than 10 nM. In some embodiments, a Kd is no more than 5 nM. In some embodiments, a Kd is no more than 1 nM.

In some embodiments, provided compounds may be assessed in a library compared to many other compounds, e.g., comprising a number of other peptide sequences. In some embodiments, such a library is a phase display library. In some embodiments, such a library is a RNA, e.g., mRNA, display library. In some embodiments, such a library is a DNA display library.

Useful related technologies include those described in US 2016/0068835, U.S. Pat. Nos. 9,410,148, 8,188,260, and 9,090,668. In at least one such test using phage libraries, peptide sequences of provided compound displayed higher binding than other sequences.

### 6 Uses, Formulation and Administration

### Pharmaceutically Acceptable Compositions

According to another embodiment, present disclosure provides a composition comprising a compound described herein or a pharmaceutically acceptable derivative thereof and a pharmaceutically acceptable carrier, adjuvant, or vehicle. In some embodiments, the present disclosure provides a pharmaceutical composition comprising a compound of the present disclosure and a pharmaceutically acceptable carrier. In some embodiments, the present disclosure provides a pharmaceutical composition comprising a therapeutically effective amount of a compound and a pharmaceutically acceptable carrier.

In some embodiments, a pharmaceutically acceptable carrier, adjuvant, or vehicle is a non-toxic carrier, adjuvant, or vehicle that does not destroy the pharmacological activity of the compound with which it is formulated. Pharmaceutically acceptable carriers, adjuvants or vehicles that may be include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some embodiments, a pharmaceutically acceptable derivative is a non-toxic salt, ester, salt of an ester or other derivative of a compound that, upon administration to a recipient, is capable of providing, either directly or indirectly, a compound or an active metabolite or residue thereof.

Compositions may be administered orally, parenterally, by inhalation spray, topically, rectally, nasally, buccally, vaginally or via an implanted reservoir. In some embodiments, parenteral administration includes subcutaneous, intravenous, intramuscular, intra-articular, intra-synovial, intrasternal, intrathecal, intrahepatic, intralesional and intracranial injection or infusion techniques. In some embodiments, compositions are administered orally, intraperitoneally or intravenously. Sterile injectable forms of compositions may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

### Uses of Compounds and Compositions Thereof

Provided technologies are useful for many CD38-associated purposes. Among other things, provided compounds and compositions thereof are useful for CD38 binding and modulation of CD38 properties and/or activities.

In some embodiments, provided technologies are particularly useful for delivering an agent to a target comprising CD38 for detection, diagnostic and/or therapeutic uses, either in vivo or in vitro. In some embodiments, such an agent is delivered by a conjugate comprising a CD38-binding moiety.

Among other things, provided compounds are useful for preventing or treating various CD38-associated conditions, disorders or diseases. In some embodiments, a condition, disorder or disease is cancer.

In some embodiments, provided compounds are useful for labeling targets, e.g., cells, having CD38 for detection, isolation, characterization or targeting by a therapeutic agent (e.g., a payload drug by a PDC).

### EXEMPLIFICATION

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein.

### Example 1. Provided Compounds Bind CD38

Phage display libraries were constructed to assess binding of a number of peptides to CD38 using technologies described in, e.g., US 2016068835, U.S. Pat. Nos. 9,410,148, and 8,188,260. Peptides comprising the following sequences were identified:

Recombinant human CD38 proteins were purchased from R&D Systems. In some embodiments, the following codons were used. ClAc-Ala: N-Chloroacetyl-L-alanine; MePhe: N-Methyl-L-phenylalanine; MeGly: N-Methyl-glycine; Ahp: (S)-2-aminoheptanoic acid; Bph: (S)-3-([1,1′-biphenyl]-4-yl)-2-aminopropanoic acid.

A number of compounds having similar structures to compound I-1 (difference being amino acid sequence) were prepared and assessed in an ELISA assay. Certain data are presented below. The first sequence is of I-1, and SEQ ID.NO.1.

TABLE 2

| | | | | | | | | | | | | | Binding |
|---|---|---|---|---|---|---|---|---|---|---|---|---|---|
| 1 | 2 | 3 | 4 | 5 | 6 | 7 | 8 | 9 | 10 | 11 | 12 | 13 | Assay* |
| A | R | Ahp | Y | H | D | G | V | L | Bph | Ahp | D | C | 1.97 |
| A | A | Ahp | Y | H | D | G | V | L | Bph | Ahp | D | C | 0.60 |
| A | R | A | Y | H | D | G | V | L | Bph | Ahp | D | C | 0.11 |
| A | R | Ahp | A | H | D | G | V | L | Bph | Ahp | D | C | 0.11 |
| A | R | Ahp | Y | A | D | G | V | L | Bpb | Ahp | D | C | 0.28 |
| A | R | Ahp | Y | H | A | G | V | L | Bph | Ahp | D | C | 0.93 |
| A | R | Ahp | Y | H | D | A | V | L | Bpb | Ahp | D | C | 1.49 |
| A | R | Ahp | Y | H | D | G | A | L | Bph | Ahp | D | C | 0.30 |
| A | R | Ahp | Y | H | D | G | V | A | Bph | Ahp | D | C | 0.08 |
| A | R | Ahp | Y | H | D | G | V | L | A | Ahp | D | C | 0.09 |
| A | R | Ahp | Y | H | D | G | V | L | Bph | A | D | C | 0.20 |
| A | R | Ahp | Y | H | D | G | V | L | Bph | Ahp | A | C | 1.79 |

*Larger values indicate stronger binding.

A number of compounds having similar structures to compound I-2 (difference being amino acid sequence) were prepared and assessed in an ELISA assay. Certain data are presented below. The first sequence is of I-2, and SEQ ID NO. 2.

TABLE 3

| | | | | | | | | | | | | Binding |
|---|---|---|---|---|---|---|---|---|---|---|---|---|
| 1 | 2 | 3 | 4 | 5 | 6 | 7 | 8 | 9 | 10 | 11 | 12 | Assay* |
| A | L | H | MeF | V | L | P | Bph | V | W | V | C | 1.69 |
| A | A | H | MeF | V | L | P | Bph | V | W | V | C | 0.84 |
| A | L | A | MeF | V | L | P | Bph | V | W | V | C | 0.37 |
| A | L | H | A | V | L | P | Bph | V | W | V | C | 0.10 |
| A | L | H | MeF | A | L | P | Bph | V | W | V | C | 0.47 |
| A | L | H | MeF | V | A | P | Bph | V | W | V | C | 0.09 |
| A | L | H | MeF | V | L | A | Bph | V | W | V | C | 0.11 |
| A | L | H | MeF | V | L | P | A | V | W | V | C | 0.09 |
| A | L | H | MeF | V | L | P | Bph | A | W | V | C | 0.12 |
| A | L | H | MeF | V | L | P | Bph | V | A | V | C | 0.10 |
| A | L | H | MeF | V | L | P | Bph | V | W | A | C | 0.41 |

* Larger values indicate stronger binding.

### Example 2. Exemplary Binding Constants

One of many useful technology for assessing CD38 binding is surface plasmon resonance (SPR). For example, the binding constant of I-1 (SEQ ID NO. 1; same as below) and I-2 (SEQ ID NO. 2; same as below) to CD38 was analyzed by a SPR assay using BIACORE T200 (cytiva: Former GE Healthcare). After the equilibration of Series S Sensor Chip CM3 (produced by cytiva: Former GE Healthcare) with Running Buffer (HBS-EP with 1% (v/v) DMSO), an EDC/NHS mixture was injected at a flow rate of 10 L/min for 4 minutes to thereby activate the functional groups on sensor chip. CD38 in 10 mM Acetate (pH5.5) was injected immobilized to at a flow rate of 5 L/min. For 4 minutes to immobilize the CD38 on the substrate surfaces of the sensor chip. Then Ethanolamine was injected at a flow rate of 10 L/min for 4 minutes. 1.0 M Ethanolamine (aq.) was injected at a flow rate of 10 L/min for 420 seconds for capping. 10 mM Peptides in DMSO were diluted to obtain 10 uM with Running Buffer, and prepared 100 nM, 50 nM, 25 nM, 10 nM, 5 nM of each peptide solutions (Peptide Samples).

Using these Peptide Samples, kinetics of peptides against CD38 was measured. The method adopted for sample measurement was a single-cycle kinetics method. The analysis was conducted using the evaluation software provided with Biacore T200. A DMSO correction curve obtained by solvent correction measurements was applied for the analysis. Kinetics fitting was done on the difference data obtained by subtracting the baseline data from sample measurement data.

KD values were calculated based on the association rate constant (ka) and dissociation rate constant (kd). The obtained results are shown in the following Table.

TABLE 4

| Compound | Ka | Kd | KD (M) |
|---|---|---|---|
| I-1 (SEQ ID NO. 1) | 7.7*10⁵ | 1.8*10⁻³ | 2.3*10⁻⁹ |
| I-2 (SEQ ID NO. 2) | 3.6*10⁵ | 1.3*10⁻² | 3.5*10⁻⁸ |

As demonstrated herein, provided compounds can effectively bind to CD38.

### Example 3. Exemplary Synthesis of Provided Compounds

Abbreviations used herein are well-known to those skilled in the art. Some of the abbreviations used are as follows: calcd. for calculated; Fmoc for fluorenylmethyloxycarbonyl; HOAt for 1-hydroxy-7-azabenzotriazole; HATU for 1-[bis(dimethylamino)methylene]-1H-1,2,3-triazolo[4,5-b]pyridinium 3-oxid hexafluorophosphate; DMSO for dimethyl sulfoxide; DMF for N,N-dimethylformamide; mL for milliliter; M for molar; v/v for volume per volume; Mpe for methylpent-3-yl (amino acids side chain protecting group); Pbf for (2,2,4,6,7-pentamethyldihydrobenzofuran-5-sulfonyl (amino acids side chain protecting group); TFA for trifluoroacetic acid; TIS for triisopropyl silane; MeF for methyl phenylalanin; Ahp for 2-amino-Heptanoic acid; BPh for beta-(4-biphenyl)-alanine. Certain useful technologies were described in Bioconjugate Chem. 2018, 29, 1847-1851. In some embodiments, peptides had ClAc- at N-terminal. In some embodiments, for cyclization, deprotected peptides were dissolved in DMSO 0.1% TFA followed by the addition of TEA until the solution was basic.

Typically, peptides were synthesized by standard solid phase Fmoc chemistry. Peptides were assembled on Biotage Syro I (Biotage Japan). Peptides with unsubstituted carboxamide on the C-terminus in the present invention were prepared from 9-Fmoc-amino-xanthen-3-yloxy-Merrifield resin (Fmoc protected Sieber resin). Certain amino acid building blocks used for the cyclic peptide derivatives in the present invention were listed below with protecting groups of the side chain as shown in parenthesis. The listed amino acid building blocks were commercially available and were used without any further purification. Fmoc-Ahp-OH; Fmoc-Ala-OH; Fmoc-Arg(Pbf)-OH; Fmoc-Asp(OMpe)-OH; Fmoc-Cys(Trt)-OH; Fmoc-Gly-OH; Fmoc-His(Boc)-OH; Fmoc-Leu-OH; Fmoc-Lys(Boc)-OH; Fmoc-Pro-OH; Fmoc-Trp(Boc)-OH; Fmoc-Tyr(tBu)-OH; Fmoc-Val-OH; Fmoc-Arg-OH; Fmoc-Glu-OH; Fmoc-Gly-OH; Fmoc-MetO2-OH; Fmoc-Hse-OH; Fmoc-Har-OH; Fmoc-D-Ala-OH; Fmoc-W6N—OH; Fmoc-Ano-OH; Fmoc-Ado-OH; Fmoc-PhNle-OH; Fmoc-PhNva-OH; Fmoc-Cit-OH; Fmoc-F3G-OH; Fmoc-FRNNMe-OH; Fmoc-RNNdMe-OH; Fmoc-RNdMe-OH; Fmoc-hCit-OH; Fmoc-4Py2NH2(Boc)-OH.

Circulation of peptides disclosed in the present inention was performed by disolving the peptide in DMSO, final concentration as 5 mM, then adding 6 equivalents of TEA and stirred for about 16 hours. The reaction solution was neutralized by Acetic Acid and then vacuum concentrated using Biotage (trademark) V-10 (Biotage Japan).

Purification of the peptides disclosed in the present invention was performed by reverse-phase HPLC, for example, using the following conditions: Stationary phase; Waters Xbridge™, C18 5 μm OBD™, 50×250 mm; Mobile phases; eluent A (0.1% TFA in H₂O), eluent B (0.1% TFA in CH₃CN). Gradients were designed based on the specific requirements of the separation problem in each sample.

The structure of peptides disclosed in the present invention was confirmed, e.g., by ESI-MS (+). Purity of the peptides described herein were determined by the analytical condition below, and its retention time was given in the delineated conditions for the characterization purpose.

### Analytical Conditions

Stationary phase: Kinetex (trademark) EVO C18 100 Angstroms, 2.6 m, 2.1×150 mm.
Mobile phases: eluent A (0.025% TFA in H₂O); and eluent B (0.025% TFA in CH₃CN).
Column temperature: 60° C.
Gradient: see in each experimental.
Detection method: UV 225 nm.
Flow rate: 0.25 mL/min.

### Abbreviation

TABLE 5

| | |
|---|---|
| Ahp | 7-Aminoheptanoic acid |
| Bph | β-(4- biphenylyl)-alanine |
| MetO2 | Methionine Sulfone |
| Hse | Homoserine |
| Har | N6-carbamimidoyl-L-lysine |
| da | D-alanine |
| W6N | (S)-2-amino-3-(1H-pyrrolo[2,3-c]pyridin-3-yl)propanoic acid |
| Ano | (S)-2-aminononanoic acid |
| Ado | (S)-2-aminoundecanoic acid |
| PhNle | (S)-2-amino-6-phenylhexanoic acid |
| PhNva | (S)-2-amino-5-phenylpentanoic acid |
| Nal2 | (2-Naphthyl)alanine |
| Cit | Citrulline |
| F3G | 3-Guanidinophenylalanine |
| RNMe | N⁵-[imino(methylamino)methyl]-acetate-L-ornithine |
| | CAS: 1135616-49-7 |
| RNNdMe | N⁵-[(methylamino)(methylimino)methyl]- L-ornithine |
| RNdMe | N⁵-[(dimethylamino)iminomethyl]- L-ornithine |
| | CAS: 1185841-84-2 |
| hCit | 2-Amino-5-(carbamoylamino) hexanoic acid |
| | CAS: 201485-17-8 |
| 4Py2NH2 | (S)-2-amino-3-(2-aminopyridin-4-yl) propanoic acid |

### Example 4. Exemplary Synthesis of I-1 (SEQ ID NO.1)

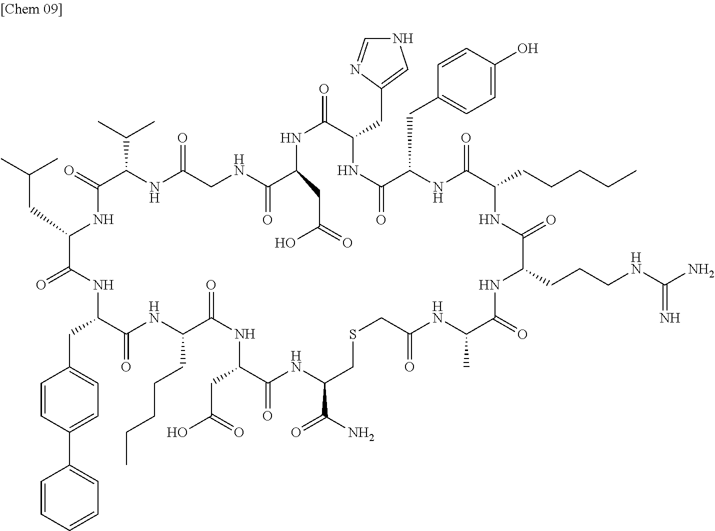 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)CNC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-4-biphenylala-OH; Fmoc-Leu-OH; Fmoc-Val-OH; Fmoc-Gly-OH; Fmoc-His(Boc)-OH; Fmoc-Tyr(tBu)-OH; Fmoc-Arg(Pbf)-OH; Fmoc-Ala-OH. MS (ESI+); calcd. for [M+2H]²⁺ 832.9, found 833.3. Gradient for the purity assessment; linear gradient, 20-60% eluent B/eluent A in 20 min. t^{ret}=8.7 min. Purity: 98.8%.

### Example 5. Exemplary Synthesis of I-2 (SEQ ID NO.2)

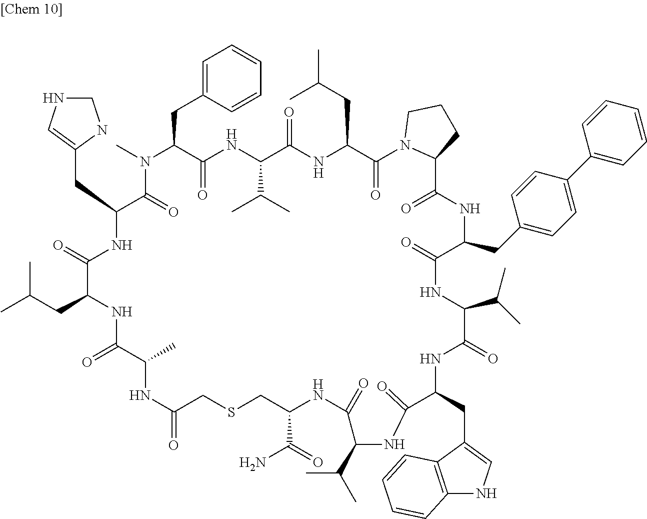 [SMILES: CC(C)C[C@@H]1NC(=O)[C@H](C)NC(=O)CSC[C@@H](C(N)=O)NC(=O)[C@H](C(C)C)NC(=O)[C@H](Cc2c[nH]c3ccccc23)NC(=O)[C@H](C(C)C)NC(=O)[C@H](Cc2ccc(-c3ccccc3)cc2)NC(=O)[C@@H]2CCCN2C(=O)[C@H](CC(C)C)NC(=O)[C@H](C(C)C)NC(=O)[C@H](Cc2ccccc2)N(C)C(=O)[C@H](CC2=CNCN2)NC1=O]

Fmoc amino acids used in the synthesis included Fmoc-Cys(Trt)-OH; Fmoc-Val-OH; Fmoc-Trp(Boc)-OH; Fmoc-4-biphenylala-OH; Fmoc-Pro-OH; Fmoc-Leu-OH; Fmoc-N-Me-Phe-OH; Fmoc-His(Boc)-OH; Fmoc-Ala-OH. MS (ESI+); calcd. for [M+2H]²⁺ 780.4, found 780.8. Gradient for the purity assessment; linear gradient, 20-60% eluent B/eluent A in 20 min. t^{ret}=8.6 min. Purity: 98.4%.

### Example 6. Exemplary Synthesis of I-3

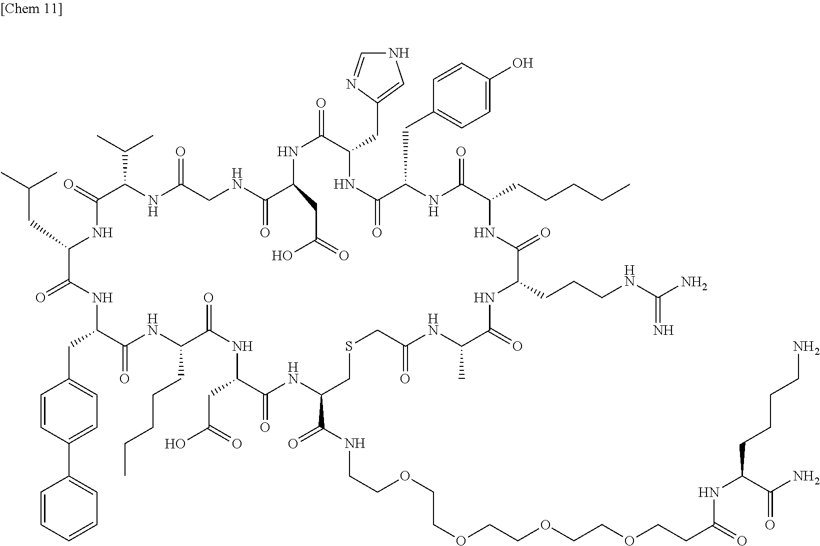 [SMILES: CCCCC[C@@H]1NC(=O)[C@H](CCCNC(=N)N)NC(=O)[C@H](C)NC(=O)CSC[C@@H](C(=O)NCCOCCOCCOCCOCCC(=O)N[C@@H](CCCCN)C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)CNC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Lys(Boc)-OH; Fmoc-PEG-OH; Fmoc-Cys(Trt)-OH; Fmoc-Asp(OMpe)-OH; Fmoc-Ahp-OH; Fmoc-4-biphenylala-OH; Fmoc-Leu-OH; Fmoc-Val-OH; Fmoc-Gly-OH; Fmoc-His(Boc)-OH; Fmoc-Tyr(tBu)-OH; Fmoc-Arg(Pbf)-OH; Fmoc-Ala-OH. MS (ESI+); calcd. for [M+2H]²⁺ 1020.5, found 1021.0. Gradient for the purity assessment; linear gradient, 20-60% eluent B/eluent A in 20 min. t^{ret}=7.9 min. Purity: 98.0%.

### Example 7. Exemplary Synthesis of I-4

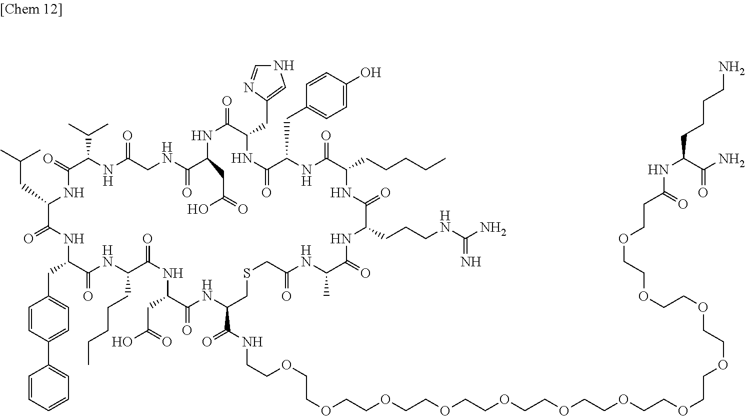 [SMILES: CCCCC[C@@H]1NC(=O)[C@H](CCCNC(=N)N)NC(=O)[C@H](C)NC(=O)CSC[C@@H](C(=O)NCCOCCOCCOCCOCCOCCOCCOCCOCCOCCOCCOCCOCCC(=O)N[C@@H](CCCCN)C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)CNC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Lys(Boc)-OH; Fmoc-PEG-OH; Fmoc-Cys(Trt)-OH; Fmoc-Asp(OMpe)-OH; Fmoc-Ahp-OH; Fmoc-4-biphenylala-OH; Fmoc-Leu-OH; Fmoc-Val-OH; Fmoc-Gly-OH; Fmoc-His(Boc)-OH; Fmoc-Tyr(tBu)-OH; Fmoc-Arg(Pbf)-OH; Fmoc-Ala-OH. MS (ESI+); calcd. for [M+2H]²⁺ 1196.6, found 1197.2. Gradient for the purity assessment; linear gradient, 20-60% eluent B/eluent A in 20 min. t^{ret}=8.6 min. Purity: 99.2%.

### Example 8. Exemplary Synthesis of I-5

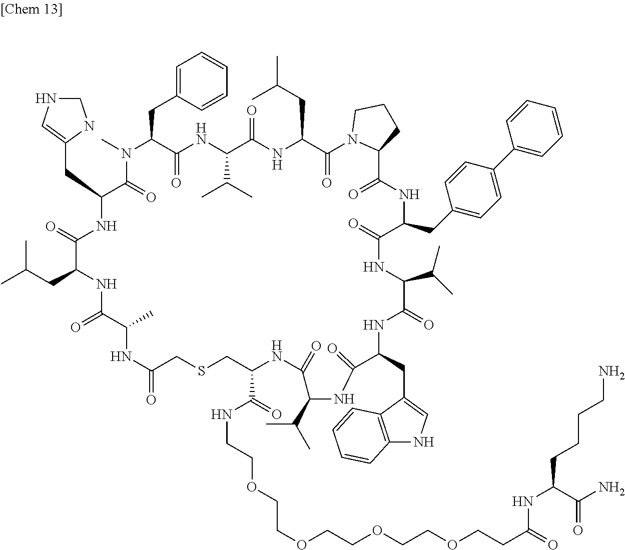 [SMILES: CC(C)C[C@@H]1NC(=O)[C@H](C)NC(=O)CSC[C@@H](C(=O)NCCOCCOCCOCCOCCC(=O)N[C@@H](CCCCN)C(N)=O)NC(=O)[C@H](C(C)C)NC(=O)[C@H](Cc2c[nH]c3ccccc23)NC(=O)[C@H](C(C)C)NC(=O)[C@H](Cc2ccc(-c3ccccc3)cc2)NC(=O)[C@@H]2CCCN2C(=O)[C@H](CC(C)C)NC(=O)[C@H](C(C)C)NC(=O)[C@H](Cc2ccccc2)N(C)C(=O)[C@H](CC2=CNCN2)NC1=O]

Fmoc amino acids used in the synthesis include Fmoc-Lys(Boc(—OH; Fmoc-PEG-OH; Fmoc-Cys(Trt)-OH; Fmoc-Val-OH; Fmoc-Trp(Boc)-OH; Fmoc-4-biphenylala-OH; Fmoc-Pro-OH; Fmoc-Leu-OH; Fmoc-N-Me-Phe-OH; Fmoc-His(Boc)-OH; Fmoc-Ala-OH. MS (ESI+); calcd. for [M+2H]²⁺ 968.0, found 968.5. Gradient for the purity assessment; linear gradient, 20-60% eluent B/eluent A in 20 min. t^{ret}=14.7 min. Purity: 98.0%.

### Example 9. Exemplary Synthesis of I-6

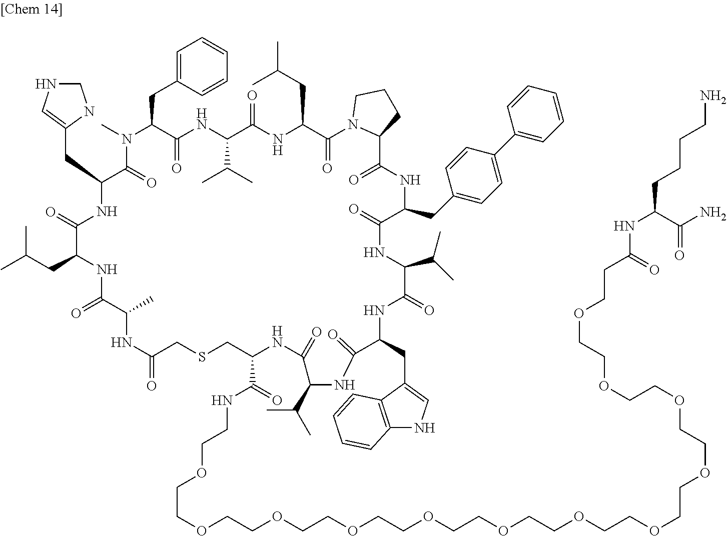 [SMILES: CC(C)C[C@@H]1NC(=O)[C@H](C)NC(=O)CSC[C@@H](C(=O)NCCOCCOCCOCCOCCOCCOCCOCCOCCOCCOCCOCCOCCC(=O)N[C@@H](CCCCN)C(N)=O)NC(=O)[C@H](C(C)C)NC(=O)[C@H](Cc2c[nH]c3ccccc23)NC(=O)[C@H](C(C)C)NC(=O)[C@H](Cc2ccc(-c3ccccc3)cc2)NC(=O)[C@@H]2CCCN2C(=O)[C@H](CC(C)C)NC(=O)[C@H](C(C)C)NC(=O)[C@H](Cc2ccccc2)N(C)C(=O)[C@H](CC2=CNCN2)NC1=O]

Fmoc amino acids used in the synthesis include Fmoc-Lys(Boc(—OH; Fmoc-PEG-OH; Fmoc-Cys(Trt)-OH; Fmoc-Val-OH; Fmoc-Trp(Boc)-OH; Fmoc-4-biphenylala-OH; Fmoc-Pro-OH; Fmoc-Leu-OH; Fmoc-N-Me-Phe-OH; Fmoc-His(Boc)-OH; Fmoc-Ala-OH. MS (ESI+); calcd. for [M+2H]²⁺ 1144.1, found 1144.7. Gradient for the purity assessment; linear gradient, 20-60% eluent B/eluent A in 20 min. t^{ret}=15.3 min. Purity; 99.5%.

### Example 10. Exemplary Synthesis of Circular Peptide Having Amino Acid Sequence Represented by SEQ ID NO.3

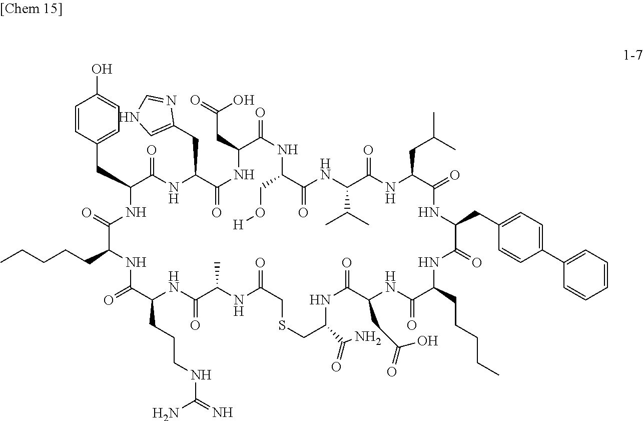 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)[C@H](CO)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-Leu-OH; Fmoc-Val-OH; Fmoc-Ser-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 847.5, found 848.4. Gradient for the purity assessment; linear gradient, 20-60% eluent B/eluent A in 20 min. t^{ret}=3.98 min. Purity: 98.6%.

### Example 11. Exemplary Synthesis of Circular Peptide Having Amino Acid Sequence Represented by SEQ ID NO.4

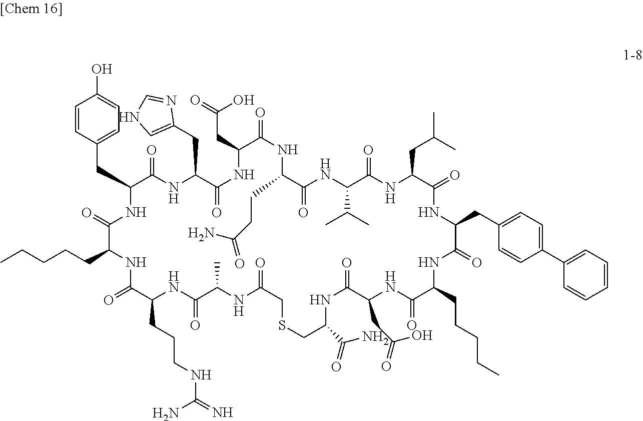 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)[C@H](CCC(N)=O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-Leu-OH; Fmoc-Val-OH; Fmoc-Gln-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 868.0, found 868.9. Gradient for the purity assessment; linear gradient, 20-60% eluent B/eluent A in 20 min. t^{ret}=3.685 min. Purity: 95.5%.

### Example 12. Exemplary Synthesis of Circular Peptide Having Amino Acid Sequence Represented by SEQ ID NO.5

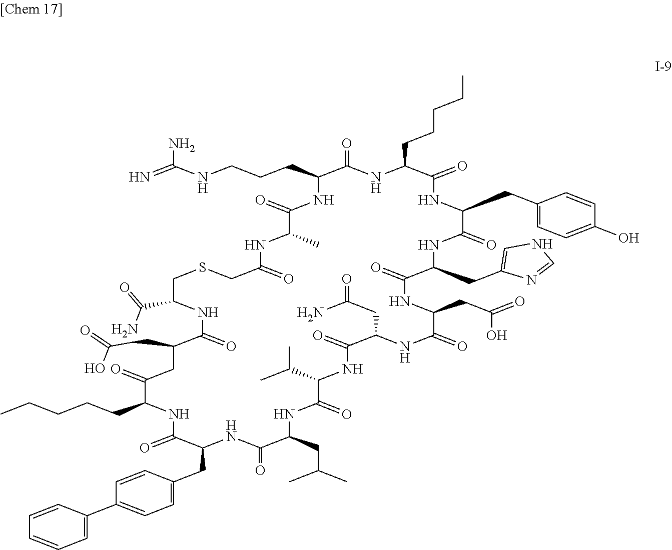 [SMILES: CCCCC[C@@H]1NC(=O)[C@H](Cc2ccc(-c3ccccc3)cc2)NC(=O)[C@H](CC(C)C)NC(=O)[C@H](C(C)C)NC(=O)[C@H](CC(N)=O)NC(=O)[C@H](CC(=O)O)NC(=O)[C@H](Cc2c[nH]cn2)NC(=O)[C@H](Cc2ccc(O)cc2)NC(=O)[C@H](CCCCC)NC(=O)[C@H](CCCNC(=N)N)NC(=O)[C@H](C)NC(=O)CSC[C@@H](C(N)=O)NC(=O)[C@H](CC(=O)O)CC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-Leu-OH; Fmoc-Val-OH; Fmoc-Asn-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 868.0, found 861.9. Gradient for the purity assessment; linear gradient, 20-60% eluent B/eluent A in 20 min. t^{ret}=3.75 min. Purity: 98.8%.

### Example 13. Exemplary Synthesis of Circular Peptide Having Amino Acid Sequence Represented by SEQ ID NO.6

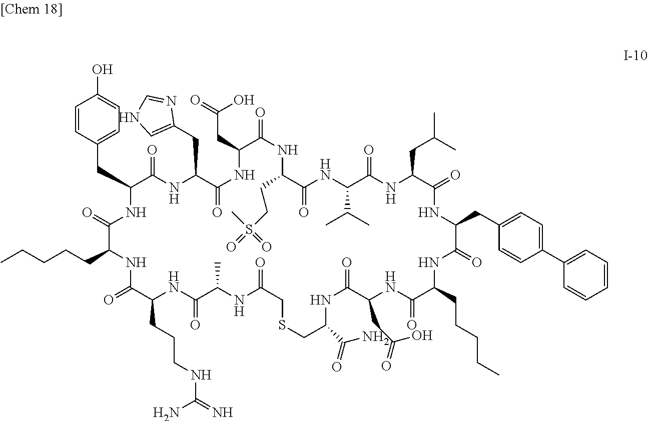 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)[C@H](CCS(C)(=O)=O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-Leu-OH; Fmoc-Val-OH; Fmoc-MetO2-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 885.5, found 886.4. Gradient for the purity assessment; linear gradient, 20-60% eluent B/eluent A in 20 min. t^{ret}=3.958 min. Purity: 97.9%.

### Example 14. Exemplary Synthesis of Circular Peptide Having Amino Acid Sequence Represented by SEQ ID NO.7

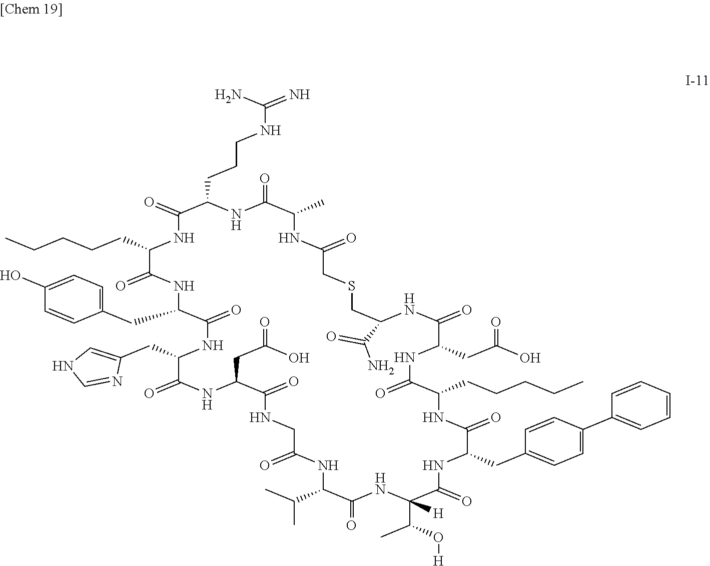 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H]([C@@H](C)O)NC(=O)[C@H](C(C)C)NC(=O)CNC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-Thr-OH; Fmoc-Val-OH; Fmoc-Gly-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 826.4, found 827.4. Gradient for the purity assessment; linear gradient, 20-60% eluent B/eluent A in 20 min. t^{ret}=3.25 min. Purity. 98.5%.

### Example 15. Exemplary Synthesis of Circular Peptide Having Amino Acid Sequence Represented by SEQ ID NO.8

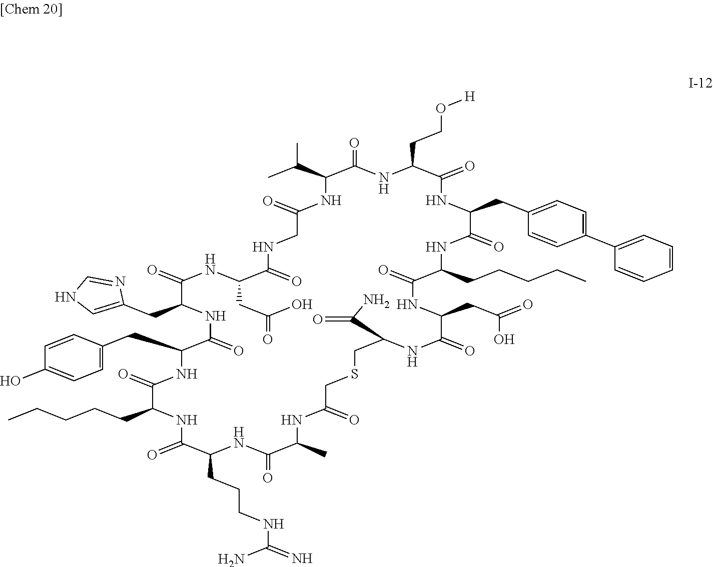 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CCO)NC(=O)[C@H](C(C)C)NC(=O)CNC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-Hse-OH; Fmoc-Val-OH; Fmoc-Gly-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 827.4, found 827.4. Gradient for the purity assessment; linear gradient, 20-60% eluent B/eluent A in 20 min. t^{ret}=3.15 min. Purity: 98.9%.

### Example 16. Exemplary Synthesis of Circular Peptide Having Amino Acid Sequence Represented by SEQ ID NO.9

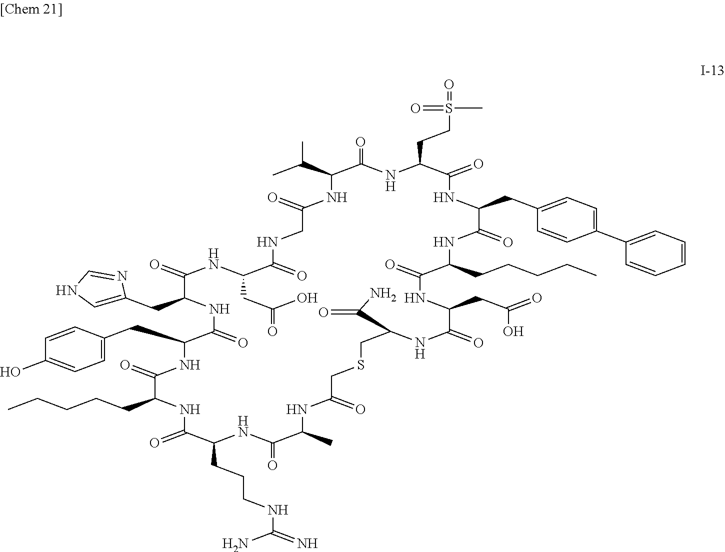 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CCS(C)(=O)=O)NC(=O)[C@H](C(C)C)NC(=O)CNC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-MetO2-OH; Fmoc-Val-OH; Fmoc-Gly-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 857.5, found 858.5. Gradient for the purity assessment; linear gradient, 20-60% eluent B/eluent A in 20 min. t^{ret}=3.22 min. Purity. 99.1%.

### Example 17. Exemplary Synthesis of Circular Peptide Having Amino Acid Sequence Represented by SEQ ID NO.10

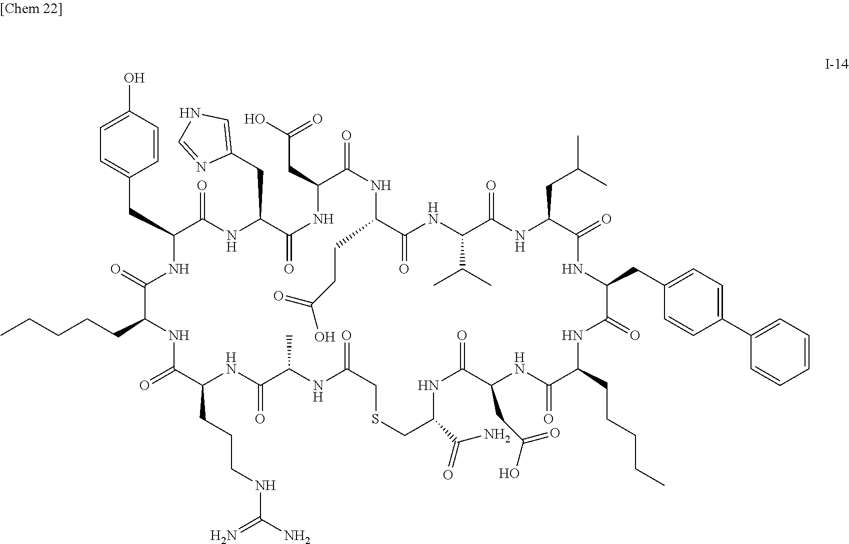 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 868.5, found 869.4. Gradient for the purity assessment; linear gradient, 20-60% eluent B/eluent A in 20 min. t^{ret}=3.93 min. Purity. 95.9%.

### Example 18. Exemplary Synthesis of Circular Peptide Having Amino Acid Sequence Represented by SEQ ID NO.11

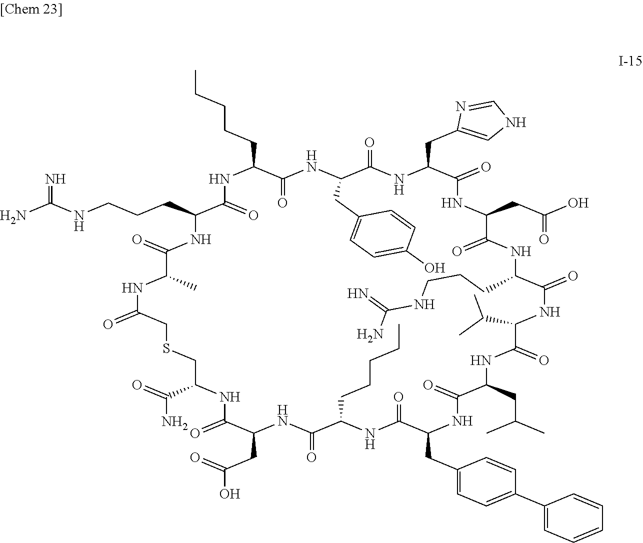 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)[C@H](CCCNC(=N)N)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 882.0, found 883.0. Gradient for the purity assessment; linear gradient, 20-60% eluent B/eluent A in 20 min. t^{ret}=3.29 min. Purity: 96.9%.

### Example 19. Exemplary Synthesis of Circular Peptide Having Amino Acid Sequence Represented by SEQ ID NO.12

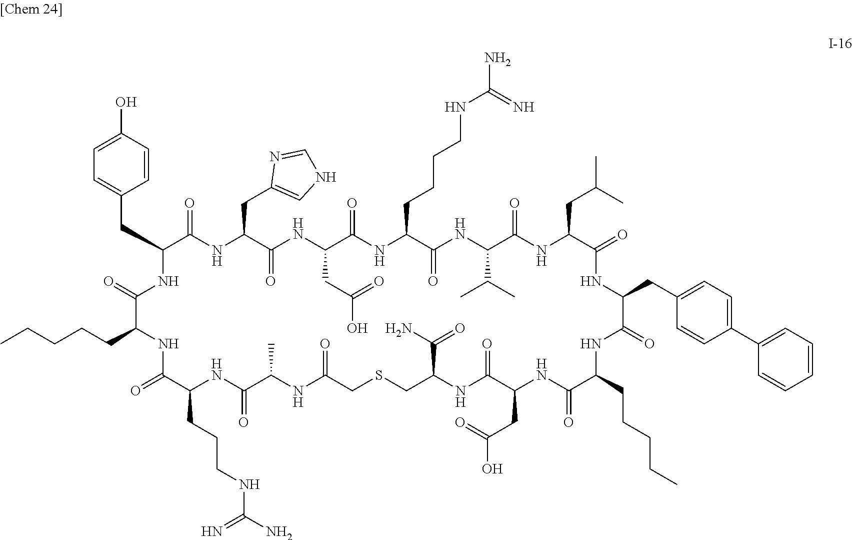 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)[C@H](CCCCNC(=N)N)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-Leu-OH; Fmoc-Val-OH; Fmoc-Har(Pbf)-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 889.1, found 890.0. Gradient for the purity assessment; linear gradient, 20-60% eluent B/eluent A in 20 min. t^{ret}=3.37 min. Purity: 96.5%.

### Example 20. Exemplary Synthesis of Circular Peptide Having Amino Acid Sequence Represented by SEQ ID NO.13

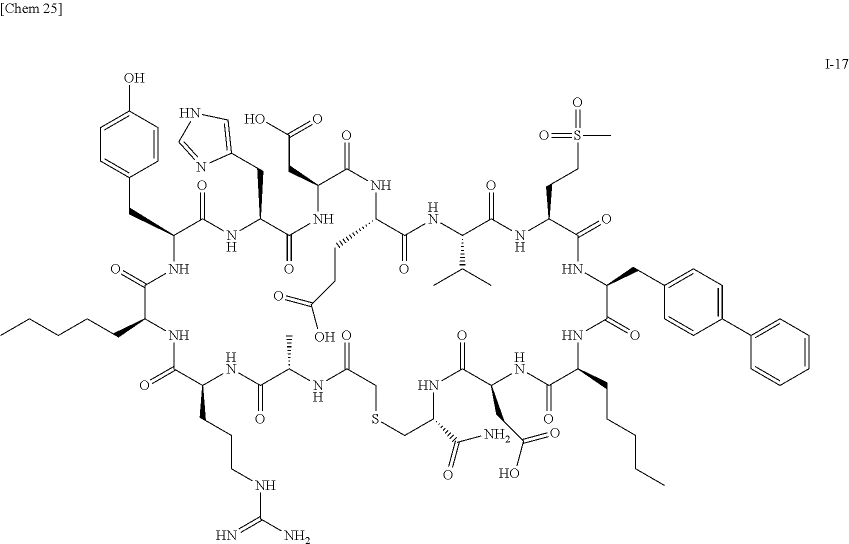 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CCS(C)(=O)=O)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-MetO2-OH; Fmoc-Val-OH; Fmoc-Gln-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 893.1, found 894.0. Gradient for the purity assessment; linear gradient, 20-60% eluent B/eluent A in 20 min. t^{ret}=3.62 min. Purity: 95.4%

### Example 21. Exemplary Synthesis of Circular Peptide Having Amino Acid Sequence Represented by SEQ ID NO.14

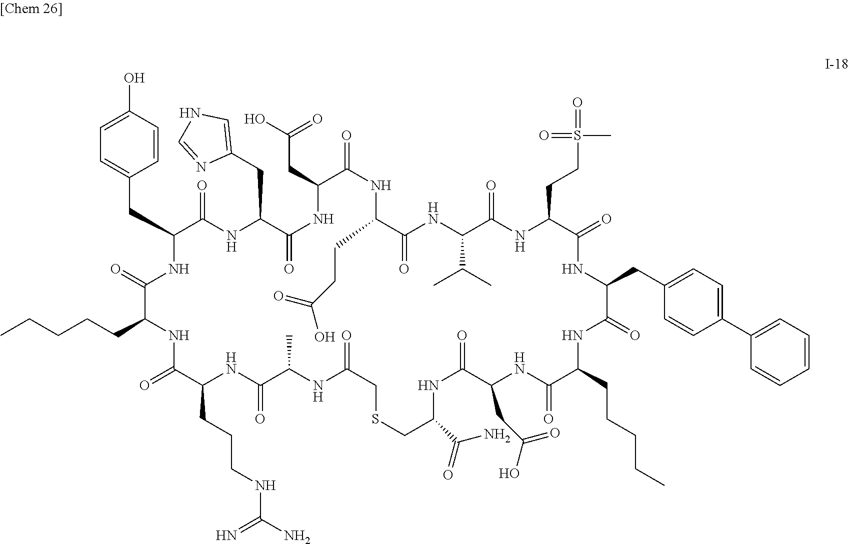 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CCS(C)(=O)=O)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-MetO2-OH; Fmoc-Val-OH; Fmoc-Glu-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 893.5, found 894.4. Gradient for the purity assessment; linear gradient, 20-60% eluent B/eluent A in 20 min. t^{ret}=3.69 min. Purity: 98.0%

### Example 22. Exemplary Synthesis of Circular Peptide Having Amino Acid Sequence Represented by SEQ ID NO.15

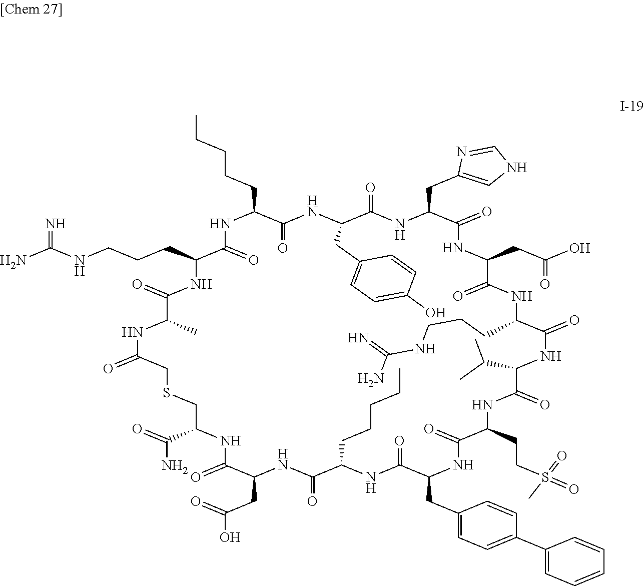 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CCS(C)(=O)=O)NC(=O)[C@H](C(C)C)NC(=O)[C@H](CCCNC(=N)N)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-MetO2-OH; Fmoc-Val-OH; Fmoc-Arg-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 907.06, found 908.0. Gradient for the purity assessment; linear gradient, 20-60% eluent B/eluent A in 20 min. t^{ret}=5.8 min. Purity: 96.8%

### Example 23. Exemplary Synthesis of Circular Peptide Having Amino Acid Sequence Represented by SEQ ID NO.16

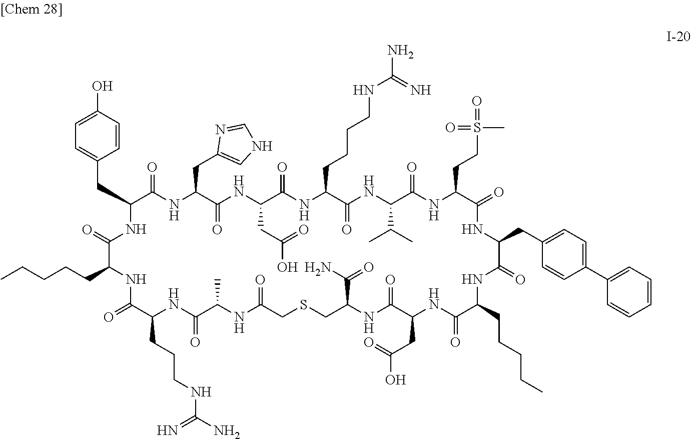 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CCS(C)(=O)=O)NC(=O)[C@H](C(C)C)NC(=O)[C@H](CCCCNC(=N)N)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-MetO2-OH; Fmoc-Val-OH; Fmoc-Har(Pbf)-OH; Fmoc-His(Boc)-OH; Fmoc-Tyr(tBu)-OH; Fmoc-Arg(Pbf)-OH; Fmoc-Ala-OH.

MS (ESI+); calcd. for [M+2H]2+ 914.1, found 915.0. Gradient for the purity assessment; linear gradient, 20-60% eluent B/eluent A in 20 min. t^{ret}=5.88 min. Purity: 96.8%

### Example 24. Exemplary Synthesis of Circular Peptide Having Amino Acid Sequence Represented by SEQ ID NO.17

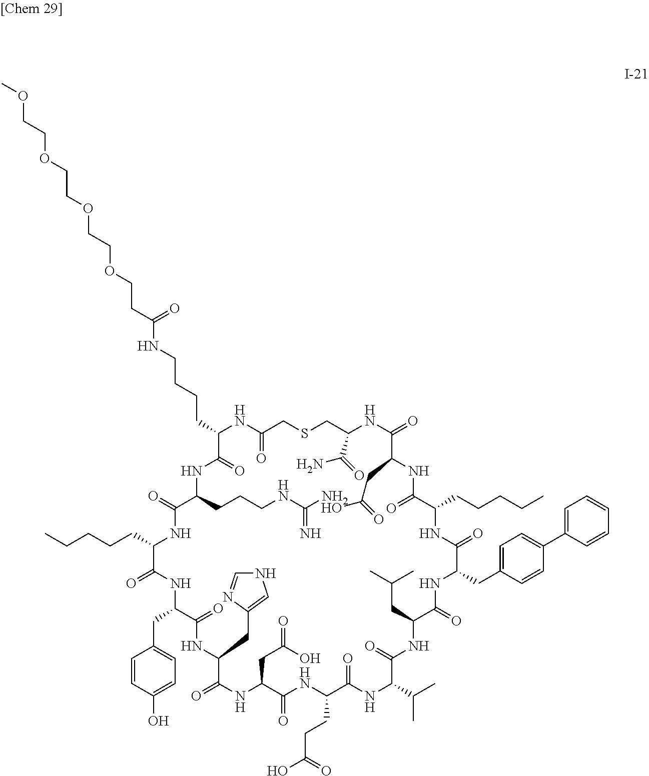 [SMILES: CCCCC[C@@H]1NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC(=O)[C@H](CCCCC)NC(=O)[C@H](CCCNC(=N)N)NC(=O)[C@H](CCCCNC(=O)CCOCCOCCOCCOC)NC(=O)CSC[C@@H](C(N)=O)NC(=O)[C@H](CC(=O)O)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Arg(Pbf)-OH; Fmoc-Lys-OH. Then side chain of the last Lys was modified through pegylation using PEG (CAS No. 1188295-19-3).

MS (ESI+); calcd. for [M+2H]2+ 1006.2, found 1006.7. Gradient for the purity assessment; linear gradient, 20-60% eluent B/eluent A in 20 min. t^{ret}=3.94 min. Purity: 91.6%

### Example 25. Exemplary Synthesis of Circular Peptide Having Amino Acid Sequence Represented by SEQ ID NO.18

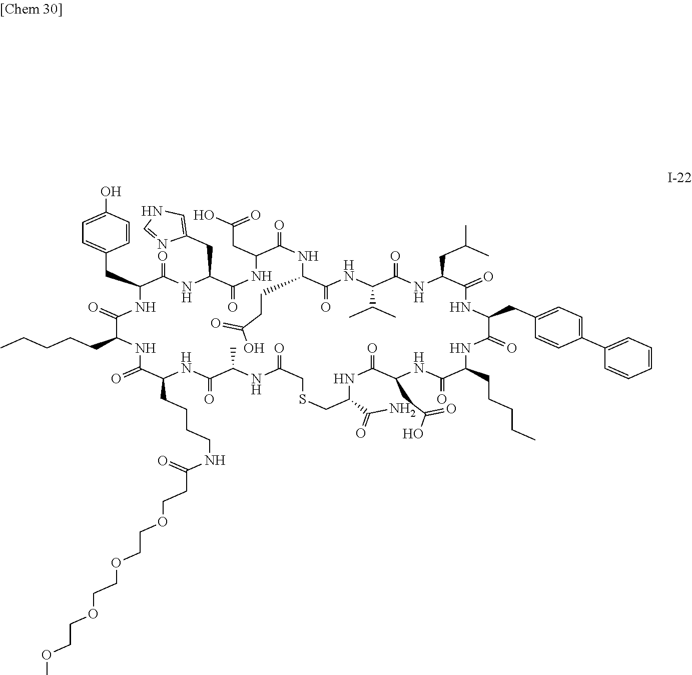 [SMILES: CCCCC[C@@H]1NC(=O)[C@H](CCCCNC(=O)CCOCCOCCOCCOC)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)C(CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Arg(Pbf)-OH; Fmoc-Lys-OH, Fmoc-Ala-OH. Then side chain of Lys at second position was modified through pegylation using PEG (CAS No. 1188295-19-3).

MS (ESI+); calcd. for [M+2H]2+ 963.6, found 964.6. Gradient for the purity assessment; linear gradient, 20-60% eluent B/eluent A in 20 min. t^{ret}=4.71 min. Purity: 92.9%

### Example 26. Exemplary Synthesis of Circular Peptide Having Amino Acid Sequence Represented by SEQ ID NO.19

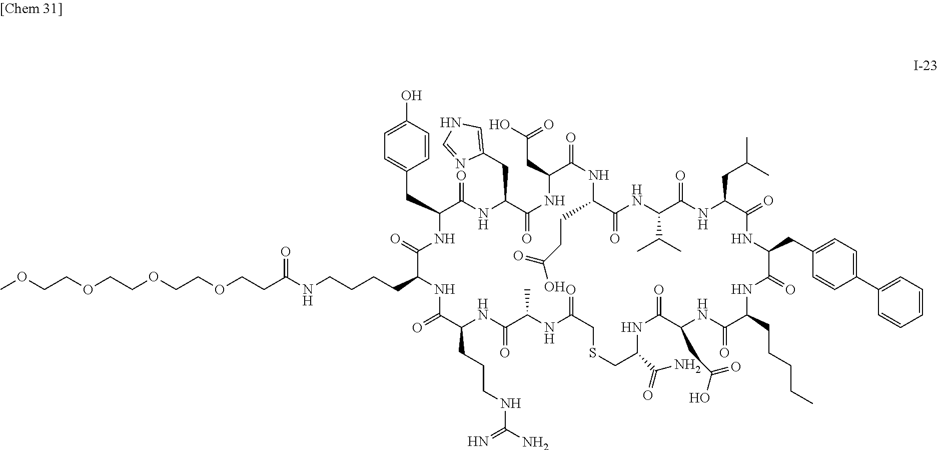 [SMILES: CCCCC[C@@H]1NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC(=O)[C@H](CCCCNC(=O)CCOCCOCCOCCOC)NC(=O)[C@H](CCCNC(=N)N)NC(=O)[C@H](C)NC(=O)CSC[C@@H](C(N)=O)NC(=O)[C@H](CC(=O)O)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Arg(Pbf)-OH; Fmoc-Lys-OH, Fmoc-Ala-OH. Then side chain of Lys at third position was modified through pegylation using PEG (CAS No. 1188295-19-3).

MS (ESI+); calcd. for [M+2H]2+ 978.1, found 979.1. Gradient for the purity assessment; linear gradient, 20-60% eluent B/eluent A in 20 min. t^{ret}=3.75 min. Purity: 86.9%

### Example 27. Exemplary Synthesis of Circular Peptide Having Amino Acid Sequence Represented by SEQ ID NO.20

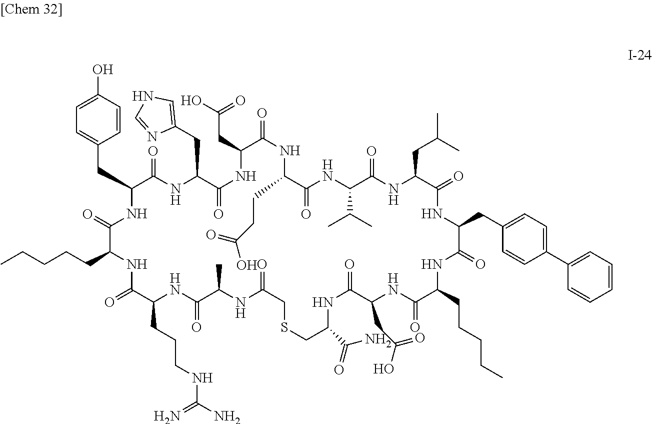 [SMILES: CCCCC[C@@H]1NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC(=O)[C@H](CCCCC)NC(=O)[C@H](CCCNC(=N)N)NC(=O)[C@@H](C)NC(=O)CSC[C@@H](C(N)=O)NC(=O)[C@H](CC(=O)O)NC1=O]

### Synthesis of SEQ ID NO.7

Fmoc amino acids used in the synthesis included Fmoc-Cys(Trt)-OH; Fmoc-Asp(OMpe)-OH; Fmoc-Ahp-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Arg(Pbf)-OH, Fmoc-D-Ala-OH.

MS (ESI+); calcd. for [M+2H]2+ 868.5, found 869.2. Gradient for the purity assessment; linear gradient, 20-60% eluent B/eluent A in 20 min. t^{ret}=4.10 min. Purity. 97.1%

### Example 28. Exemplary Synthesis of Circular Peptide Having Amino Acid Sequence Represented by SEQ ID NO.21

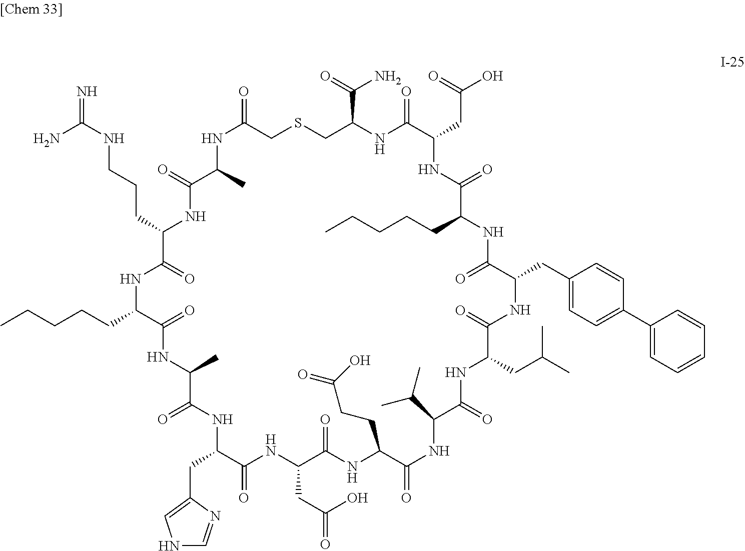 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](C)NC1=O]

Fmoc amino acids used in the synthesis included Fmoc-Cys(Trt)-OH; Fmoc-Asp(OMpe)-OH; Fmoc-Ahp-OH; Fmoc-Bph-OH; Fmoc-Leu-OH; Fmoc-Val-OH; Fmoc-Glu-OH; Fmoc-His(Boc)-OH; Fmoc-Ala-OH; Fmoc-Arg(Pbf)-OH.

MS (ESI+); calcd. for [M+2H]2+ 822.5, found 823.2. Gradient for the purity assessment; linear gradient, 20-60% eluent B/eluent A in 20 min. t^{ret}=3.96 min. Purity. 95.0%

### Example 29. Exemplary Synthesis of Circular Peptide Having Amino Acid Sequence Represented by SEQ ID NO.22

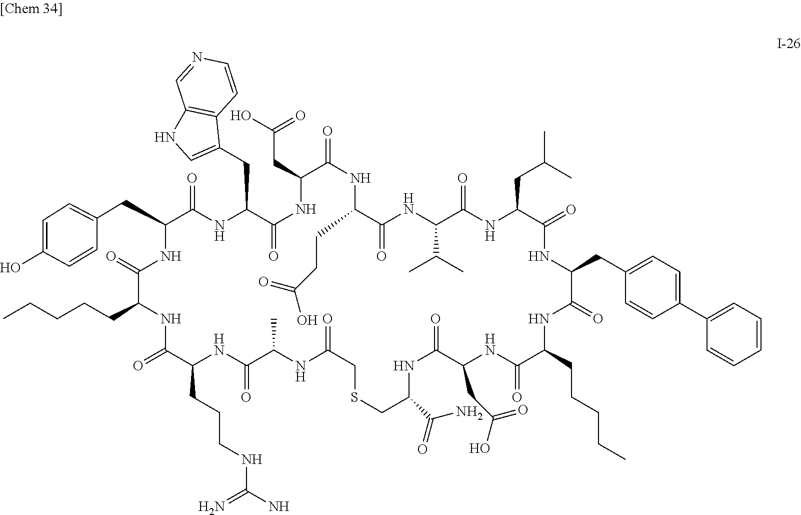 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCC)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]c3cnccc23)NC(=O)[C@H](Cc2ccc(O)cc2)NC1=O]

moc amino acids used in the synthesis included Fmoc-Cys(Trt)-OH; Fmoc-Asp(OMpe)-OH; Fmoc-Ahp-OH; Fmoc-Bph-OH; Fmoc-Leu-OH; Fmoc-Val-OH; Fmoc-Glu-OH; Fmoc-W6N—OH; Fmoc-Ala-OH; Fmoc-Arg(Pbf)-OH.

MS (ESI+); calcd. for [M+2H]2+ 893.5, found 894.4. Gradient for the purity assessment; linear gradient, 20-60% eluent B/eluent A in 20 min. t^{ret}=3.67 min. Purity: 97.3%

### Example 30. Exemplary Synthesis of Circular Peptide Having Amino Acid Sequence Represented by SEQ ID NO.23

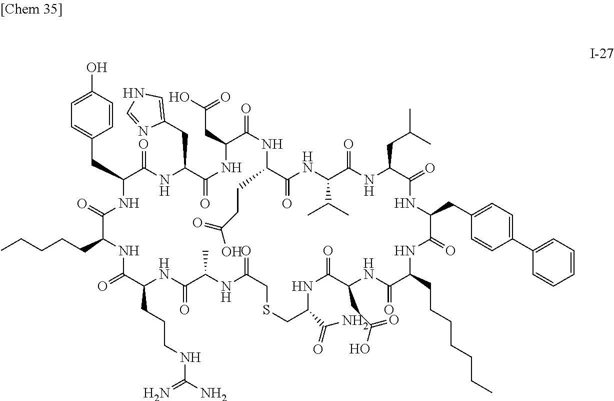 [SMILES: CCCCCCC[C@@H]1NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC(=O)[C@H](CCCCC)NC(=O)[C@H](CCCNC(=N)N)NC(=O)[C@H](C)NC(=O)CSC[C@@H](C(N)=O)NC(=O)[C@H](CC(=O)O)NC1=O]

Fmoc amino acids used in the synthesis included Fmoc-Cys(Trt)-OH; Fmoc-Ahp-OH; Fmoc-Asp(OMpe)-OH; Fmoc-Ano-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Ala-OH; Fmoc-Arg(Pbf)-OH.

MS (ESI+); calcd. for [M+2H]2+ 882.5, found 883.2. Gradient for the purity assessment; linear gradient, 20-60% eluent B/eluent A in 20 min. t^{ret}=4.38 min. Purity. 94.7%

### Example 31. Exemplary Synthesis of Circular Peptide Having Amino Acid Sequence Represented by SEQ ID NO.24

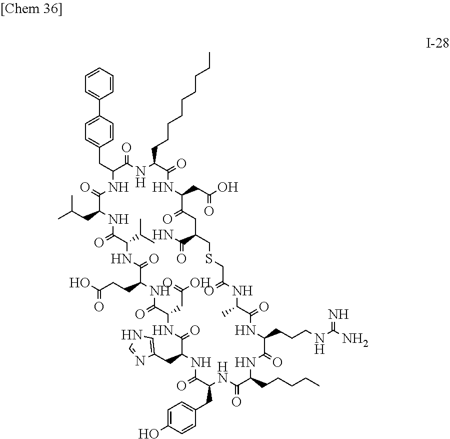 [SMILES: CCCCCCCCC[C@@H]1NC(=O)C(Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC(=O)[C@H](CCCCC)NC(=O)[C@H](CCCNC(=N)N)NC(=O)[C@H](C)NC(=O)CSC[C@@H](C(N)=O)CC(=O)[C@H](CC(=O)O)NC1=O]

Fmoc amino acids used in the synthesis included Fmoc-Cys(Trt)-OH; Fmoc-Ahp-OH; Fmoc-Asp(OMpe)-OH; Fmoc-Ado-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Ala-OH; Fmoc-Arg(Pbf)-OH.

MS (ESI+); calcd. for [M+2H]2+ 896.6, found 897.5. Gradient for the purity assessment; linear gradient, 20-60% eluent B/eluent A in 20 min. t^{ret}=4.97 min. Purity: 88.7%

### Example 32. Exemplary Synthesis of Circular Peptide Having Amino Acid Sequence Represented by SEQ ID NO.25

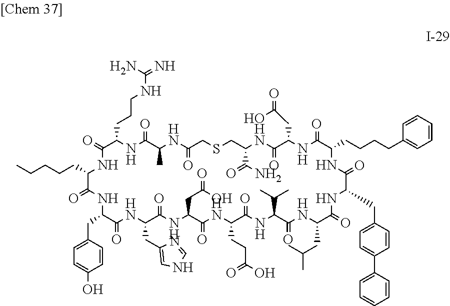 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCCc2ccccc2)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Ahp-OH; Fmoc-Asp(OMpe)-OH; Fmoc-PhNle-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Ala-OH; Fmoc-Arg(Pbf)-OH.

MS (ESI+); calcd. for [M+2H]2+ 899.5 found 900.2. Gradient for the purity assessment; linear gradient, 20-60% eluent B/eluent A in 20 min. t^{ret}=4.28 min. Purity: 97.1%

### Example 33. Exemplary Synthesis of Circular Peptide Having Amino Acid Sequence Represented by SEQ ID NO.26

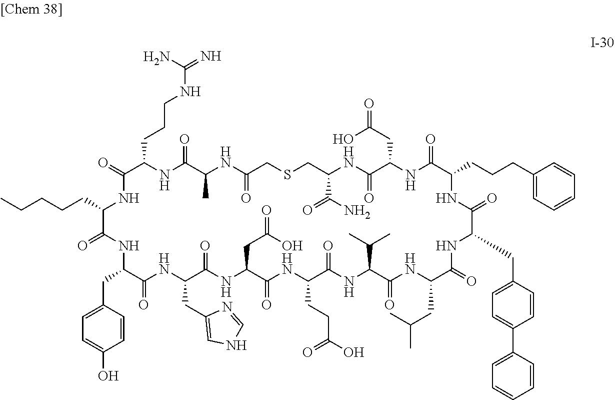 [SMILES: CCCCC[C@@H]1NC(=O)[C@H](CCCNC(=N)N)NC(=O)[C@H](C)NC(=O)CSC[C@@H](C(N)=O)NC(=O)[C@H](CC(=O)O)NC(=O)[C@H](CCCc2ccccc2)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cys(Trt)-OH; Fmoc-Ahp-OH; Fmoc-Asp(OMpe)-OH; Fmoc-PhNva-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Ala-OH; Fmoc-Arg(Pbf)-OH.

MS (ESI+); calcd. for [M+2H]2+ 892.5 found 893.2. Gradient for the purity assessment; linear gradient, 20-60% eluent B/eluent A in 20 min. t^{ret}=4.05 min. Purity: 95.8%

### Example 34. Exemplary Synthesis of SEQ ID NO.27

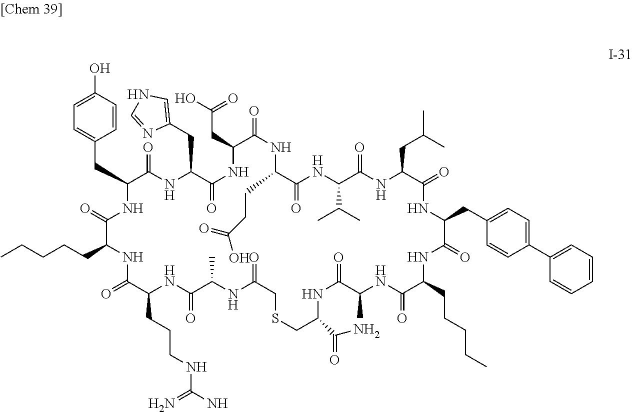 [SMILES: CCCCC[C@@H]1NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC(=O)[C@H](CCCCC)NC(=O)[C@H](CCCNC(=N)N)NC(=O)[C@H](C)NC(=O)CSC[C@@H](C(N)=O)NC(=O)[C@H](C)NC1=O]

Fmoc amino acids used in the synthesis included Fmoc-Cys(Trt)-OH; Fmoc-Ahp-OH; Fmoc-Asp(OMpe)-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Ala-OH; Fmoc-Arg(Pbf)-OH.

MS (ESI+); calcd. for [M+2H]2+ 846.5 found 847.4. Gradient for the purity assessment; linear gradient, 20-60% eluent B/eluent A in 20 min. t^{ret}=4.05 min. Purity: 91.0%

### Example 35. Exemplary Synthesis of Circular Peptide Having Amino Acid Sequence Represented by SEQ ID NO.28

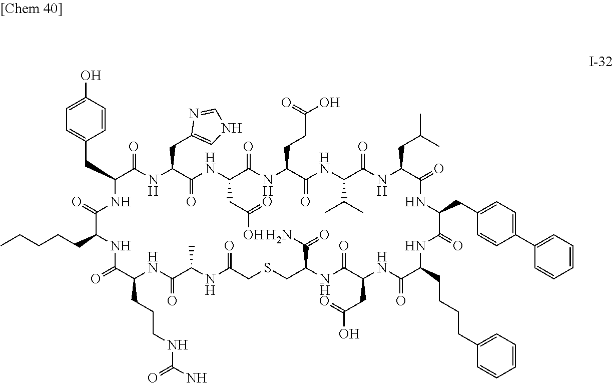 [SMILES: CCCCC[C@@H]1NC(=O)[C@H](CCCNC(N)=O)NC(=O)[C@H](C)NC(=O)CSC[C@@H](C(N)=O)NC(=O)[C@H](CC(=O)O)NC(=O)[C@H](CCCCc2ccccc2)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-Cit-OH; Fmoc-Ahp-OH; Fmoc-Cys(Trt)-OH; Fmoc-Asp(OMpe)-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Ala-OH.

MS (ESI+); calcd. for [M+2H]2+ 900.04, found 900.9. Gradient for the purity assessment; linear gradient, 20-60% eluent B/eluent A in 20 min. t^{ret}=4.85 min. Purity: 93.6%.

### Example 36. Exemplary Synthesis of Circular Peptide Having Amino Acid Sequence Represented by SEQ ID NO.29

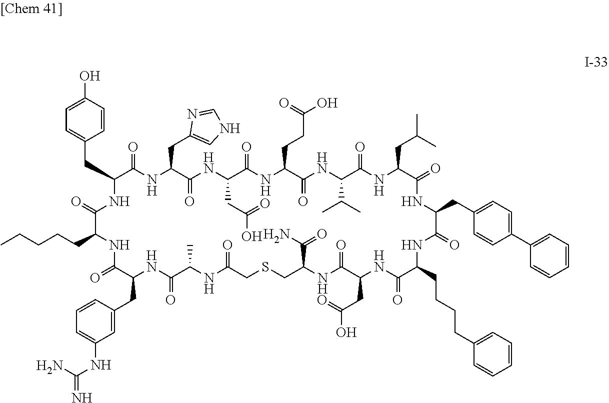 [SMILES: CCCCC[C@@H]1NC(=O)[C@H](Cc2cccc(NC(=N)N)c2)NC(=O)[C@H](C)NC(=O)CSC[C@@H](C(N)=O)NC(=O)[C@H](CC(=O)O)NC(=O)[C@H](CCCCc2ccccc2)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-F3G-OH; Fmoc-Ahp-OH; Fmoc-Cys(Trt)-OH; Fmoc-Asp(OMpe)-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Ala-OH.

MS (ESI+); calcd. for [M+2H]2+ 923.6, found 924.3. Gradient for the purity assessment; linear gradient, 20-60% eluent B/eluent A in 20 min. t^{ret}=3.95 min. Purity: 97.1%.

### Example 37. Exemplary Synthesis of Circular Peptide Having Amino Acid Sequence Represented by SEQ ID NO.30

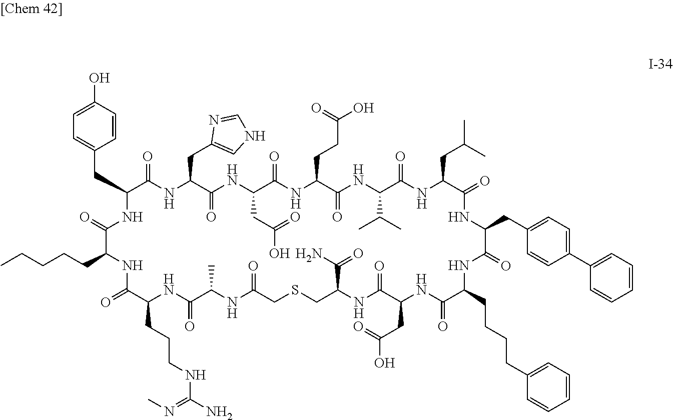 [SMILES: CCCCC[C@@H]1NC(=O)[C@H](CCCN/C(N)=N\C)NC(=O)[C@H](C)NC(=O)CSC[C@@H](C(N)=O)NC(=O)[C@H](CC(=O)O)NC(=O)[C@H](CCCCc2ccccc2)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-FRNNMe-OH; Fmoc-Ahp-OH; Fmoc-Cys(Trt)-OH; Fmoc-Asp(OMpe)-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Ala-OH.

MS (ESI+); calcd. for [M+2H]2+ 906.6, found 907.3. Gradient for the purity assessment; linear gradient, 20-60% eluent B/eluent A in 20 min. t^{ret}=4.16 min. Purity: 94.1%.

### Example 38. Exemplary Synthesis of Circular Peptide Having Amino Acid Sequence Represented by SEQ ID NO.31

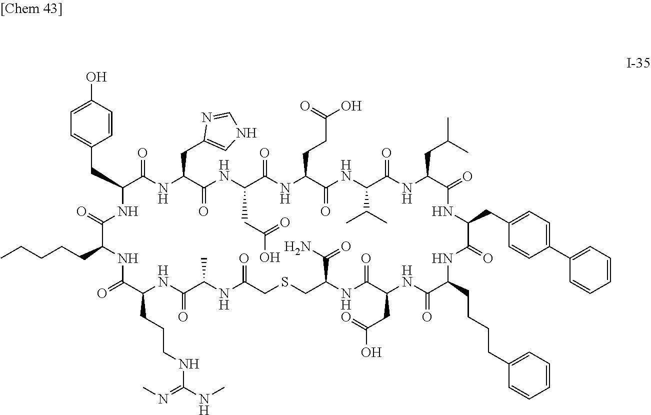 [SMILES: CCCCC[C@@H]1NC(=O)[C@H](CCCN/C(=N\C)NC)NC(=O)[C@H](C)NC(=O)CSC[C@@H](C(N)=O)NC(=O)[C@H](CC(=O)O)NC(=O)[C@H](CCCCc2ccccc2)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-RNNdMe-OH; Fmoc-Ahp-OH; Fmoc-Cys(Trt)-OH; Fmoc-Asp(OMpe)-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Ala-OH.

MS (ESI+); calcd. for [M+2H]2+ 913.6, found 914.63. Gradient for the purity assessment; linear gradient, 20-60% eluent B/eluent A in 20 min. t^{ret}=3.33 min. Purity: 94.9%.

### Example 39. Exemplary Synthesis of Circular Peptide Having Amino Acid Sequence Represented by SEQ ID NO.32

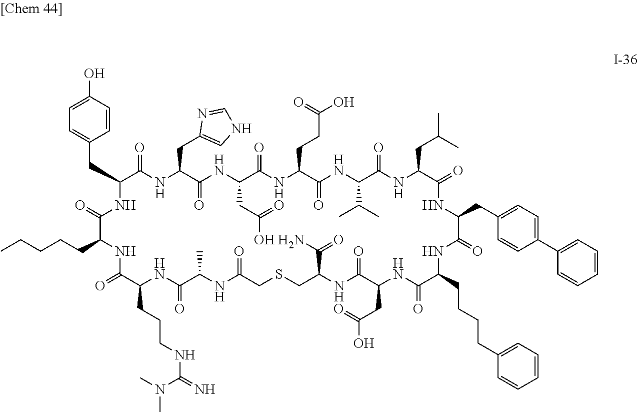 [SMILES: CCCCC[C@@H]1NC(=O)[C@H](CCCNC(=N)N(C)C)NC(=O)[C@H](C)NC(=O)CSC[C@@H](C(N)=O)NC(=O)[C@H](CC(=O)O)NC(=O)[C@H](CCCCc2ccccc2)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-RNdMe-OH; Fmoc-Ahp-OH; Fmoc-Cys(Trt)-OH; Fmoc-Asp(OMpe)-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Ala-OH.

MS (ESI+); calcd. for [M+2H]2+ 913.6, found 914.2. Gradient for the purity assessment; linear gradient, 20-60% eluent B/eluent A in 20 min. t^{ret}=4.40 min. Purity: 96.9%.

### Example 40. Exemplary Synthesis of Circular Peptide Having Amino Acid Sequence Represented by SEQ ID NO.33

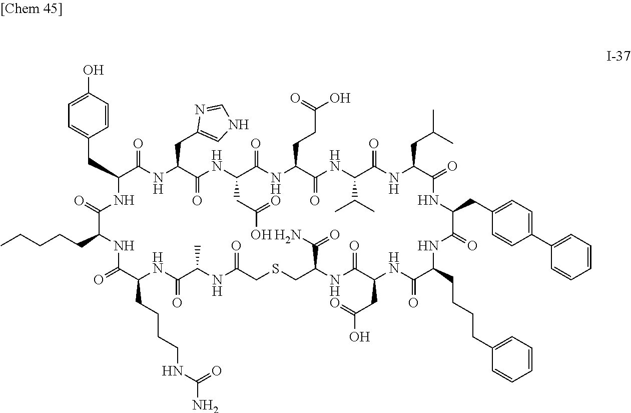 [SMILES: CCCCC[C@@H]1NC(=O)[C@H](CCCCNC(N)=O)NC(=O)[C@H](C)NC(=O)CSC[C@@H](C(N)=O)NC(=O)[C@H](CC(=O)O)NC(=O)[C@H](CCCCc2ccccc2)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-hCit-OH; Fmoc-Ahp-OH; Fmoc-Cys(Trt)-OH; Fmoc-Asp(OMpe)-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Ala-OH.

MS (ESI+); calcd. for [M+2H]2+ 907.4, found 907.9. Gradient for the purity assessment; linear gradient, 20-60% eluent B/eluent A in 20 min. t^{ret}=5.01 min. Purity: 96.9%.

### Example 41. Exemplary Synthesis of Circular Peptide Having Amino Acid Sequence Represented by SEQ ID NO.34

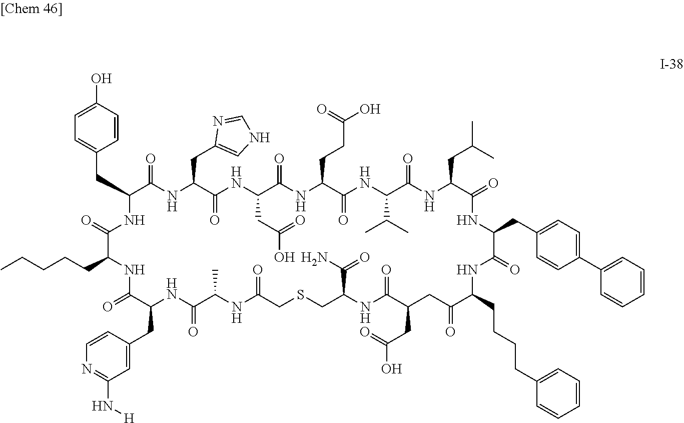 [SMILES: CCCCC[C@@H]1NC(=O)[C@H](Cc2ccnc(N)c2)NC(=O)[C@H](C)NC(=O)CSC[C@@H](C(N)=O)NC(=O)[C@H](CC(=O)O)CC(=O)[C@H](CCCCc2ccccc2)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC1=O]

Fmoc amino acids used in the synthesis included Fmoc-4Py2NH2(Boc)-OH; Fmoc-Ahp-OH; Fmoc-Cys(Trt)-OH; Fmoc-Asp(OMpe)-OH; Fmoc-Bph-OH; Fmoc-Leu-OH; Fmoc-Val-OH; Fmoc-Glu-OH; Fmoc-His(Boc)-OH; Fmoc-Tyr(tBu)-OH; Fmoc-Ala-OH.

MS (ESI+); calcd. for [M+2H]2+ 903.0, found 903.9. Gradient for the purity assessment; linear gradient, 20-60% eluent B/eluent A in 20 min. t^{ret}=3.95 min. Purity: 98.4%.

### Example 42. Binding Constant of Pegylated Compounds

The binding constant of pegylated compounds was analyzed by a surface plasmon resonance as in the Example −2. KD for I-3, I-4, I-5 and I-6 was 3.5*10⁻⁹, 4.3*10⁻⁹, 10.5*10⁻⁹, 11.7*10⁻⁹ M, respectively. As demonstrated, provided compounds which include CD38-binding peptides bound to linkers can bind strongly to CD38.

### Interaction Analysis by SPR was Performed Using Biacore (Cytiva: Former GE Healthcare).

### Interaction Analysis of SEQ ID NO.1 Variants was Performed as Follows.

SPR assay was performed using Biacore S200 (Cytiva: Former GE Healthcare).

After the equilibration of Series S Sensor Chip NTA (produced by Cytiva: Former GE Healthcare) with Running Buffer (10 mM HEPES pH7.4, 150 mM NaCl, 0.05% Tween 20), followed by injection of 350 mM EDTA at a flow rate of 10 L/min for 60 seconds and 0.5 uM NiCl2 at a flow rate of 10 L/min for 60 seconds, then 3 mM EDTA at a flow rate of 10 L/min for 120 seconds, sequently, to wash the sensor chip.

EDC/NHS mixture was injected at a flow rate of 10 L/min for 7 minutes to thereby activate the functional groups on sensor chip. His-tagged CD38 in Running Buffer (10 mM HEPES pH7.4, 150 mM NaCl, 0.05% Tween 20) was injected immobilized to at a flow rate of 10 L/min. For 7 minutes to immobilize the CD38 on the substrate surfaces of the sensor chip. Then Ethanolamine was injected at a flow rate of 10 L/min for 7 minutes.

1.0 M Ethanolamine(aq.) was injected at a flow rate of 10 L/min for 420 seconds for capping. 10 mM peptides (which were synthesised described above) in DMSO were diluted to obtain 10 uM with Running Buffer, and prepared 100 nM, 50 nM, 25 nM, 10 nM, 5 nM of each peptide's solutions (Peptide Samples).

Using these Peptide Samples, kinetics of peptides against His-tagged CD38 was measured. The method adopted for sample measurement was a single-cycle kinetics method. The analysis was conducted using the evaluation software 1.1 provided with Biacore S200. A DMSO correction curve obtained by solvent correction measurements was applied for the analysis. Kinetics fitting was done on the difference data obtained by subtracting the baseline data from sample measurement data.

KD values were calculated based on the association rate constant (ka) and dissociation rate constant (kd). The obtained results are shown in the following Tables. As demonstrated herein, provided compounds can effectively bind to CD38.

TABLE 6

| SEQ | | | | | | | | | | | | | |
|---|---|---|---|---|---|---|---|---|---|---|---|---|---|
| No. | 1 | 2 | 3 | 4 | 5 | 6 | 7 | 8 | 9 | 10 | 11 | 12 | 13 |
| 3 | A | R | Ahp | Y | H | D | S | V | L | Bph | Aph | D | C |
| 4 | A | R | Ahp | Y | H | D | Q | V | L | Bph | Aph | D | C |
| 5 | A | R | Ahp | Y | H | D | N | V | L | Bph | Aph | D | C |
| 6 | A | R | Ahp | Y | H | D | MetO2 | V | L | Bph | Aph | D | C |
| 7 | A | R | Ahp | Y | H | D | G | V | T | Bph | Aph | D | C |
| 8 | A | R | Ahp | Y | H | D | G | V | Hse | Bph | Aph | D | C |
| 9 | A | R | Ahp | Y | H | D | G | V | MetO2 | Bph | Aph | D | C |
| 10 | A | R | Ahp | Y | H | D | E | V | L | Bph | Aph | D | C |
| 11 | A | R | Ahp | Y | H | D | R | V | L | Bph | Aph | D | C |
| 12 | A | R | Ahp | Y | H | D | Har | V | L | Bph | Aph | D | C |
| 13 | A | R | Ahp | Y | H | D | Q | V | MetO2 | Bph | Aph | D | C |
| 14 | A | R | Ahp | Y | H | D | E | V | MetO2 | Bph | Aph | D | C |
| 15 | A | R | Ahp | Y | H | D | R | V | MetO2 | Bph | Aph | D | C |
| 16 | A | R | Ahp | Y | H | D | Har | V | MetO2 | Bph | Aph | D | C |
| 17 | K(MePEG4c) | R | Ahp | Y | H | D | E | V | L | Bph | Aph | D | C |
| 18 | A | K(MePEG4c) | Ahp | Y | H | D | E | V | L | Bph | Aph | D | C |
| 19 | A | R | K(MePEG4c) | Y | H | D | E | V | L | Bph | Aph | D | C |
| 20 | da | R | Ahp | Y | H | D | E | V | L | Bph | Aph | D | C |
| 21 | A | R | Ahp | A | H | D | E | V | L | Bph | Aph | D | C |
| 22 | A | R | Ahp | Y | W6N | D | E | V | L | Bph | Aph | D | C |
| 23 | A | R | Ahp | Y | H | D | E | V | L | Bph | Ano | D | C |
| 24 | A | R | Ahp | Y | H | D | E | V | L | Bph | Ado | D | C |
| 25 | A | R | Ahp | Y | H | D | E | V | L | Bph | PhNle | D | C |
| 26 | A | R | Ahp | Y | H | D | E | V | L | Bph | PhNva | D | C |
| 27 | A | R | Ahp | Y | H | D | E | V | L | Bph | Aph | A | C |
| 28 | A | Cit | Ahp | Y | H | D | E | V | L | Bph | Aph | D | C |
| 29 | A | F3G | Ahp | Y | H | D | E | V | L | Bph | Aph | D | C |
| 30 | A | RNNMe | Ahp | Y | H | D | E | V | L | Bph | Aph | D | C |
| 31 | A | RNNdMe | Ahp | Y | H | D | E | V | L | Bph | Aph | D | C |
| 32 | A | RNdMe | Ahp | Y | H | D | E | V | L | Bph | Aph | D | C |
| 33 | A | hCit | Ahp | Y | H | D | E | V | L | Bph | Aph | D | C |
| 34 | A | 4Py2NH2 | Ahp | Y | H | D | E | V | L | Bph | Aph | D | C |

TABLE 7

| SEQ | | | |
|---|---|---|---|
| ID | | | |
| NO. | Ka | Kd | KD (M) |
| 3 | 6.02*10⁵ | 2.11*10⁻³ | 3.50*10⁻⁹ |
| 4 | 5.33*10⁵ | 2.30*10⁻³ | 4.32*10⁻⁹ |
| 5 | 7.66*10⁵ | 1.61*10⁻³ | 2.11*10⁻⁹ |
| 6 | 6.31*10⁵ | 1.86*10⁻³ | 2.94*10⁻⁹ |
| 7 | 8.19*10⁵ | 5.35*10⁻² | 6.53*10⁻⁸ |
| 8 | 7.90*10⁵ | 3.06*10⁻² | 3.88*10⁻⁸ |
| 9 | 6.64*10⁵ | 6.27*10⁻³ | 9.44*10⁻⁹ |
| 10 | 4.71*10⁵ | 2.23*10⁻³ | 4.73*10⁻⁹ |
| 11 | 1.6*10⁶ | 1.9*10⁻³ | 1.19*10⁻⁹ |
| 12 | 6.6*10⁵ | 1.5*10⁻³ | 2.29*10⁻⁹ |
| 13 | 1.1*10⁶ | 2.4*10⁻² | 2.22*10⁻⁹ |
| 14 | 5.7*10⁵ | 2.2*10⁻² | 3.80*10⁻⁸ |
| 15 | 1.4*10⁶ | 1.3*10⁻² | 9.42*10⁻⁹ |
| 16 | 8.7*10⁵ | 1.5*10⁻² | 1.73*10⁻⁸ |
| 17 | 2.1*10⁵ | 9.1*10⁻⁴ | 4.37*10⁻⁹ |
| 18 | 2.2*10⁵ | 6.0*10⁻³ | 2.77*10⁻⁸ |
| 19 | 1.6*10⁵ | 1.2*10⁻³ | 7.16*10⁻⁹ |
| 20 | 7.3*10⁵ | 7.7*10⁻³ | 1.06*10⁻⁸ |
| 21 | 3.1*10⁵ | 1.6*10⁻² | 5.09*10⁻⁸ |
| 22 | 7.9*10⁵ | 8.7*10⁻³ | 1.09*10⁻⁸ |
| 23 | 8.2*10⁵ | 2.7*10⁻³ | 3.34*10⁻⁹ |
| 24 | 3.7*10⁵ | 1.3*10⁻³ | 3.53*10⁻⁹ |
| 25 | 6.3*10⁵ | 8.7*10⁻⁴ | 1.38*10⁻⁹ |
| 26 | 1.9*10⁶ | 5.0*10⁻² | 2.60*10⁻⁸ |
| 27 | 5.6*10⁵ | 2.8*10⁻³ | 4.99*10⁻⁹ |
| 28 | 6.8*10⁵ | 1.6*10⁻³ | 2.4*10⁻⁹ |
| 29 | 3.8*10⁵ | 7.8*10⁻⁴ | 2.0*10⁻⁹ |
| 30 | 3.5*10⁵ | 1.1*10⁻³ | 3.1*10⁻⁹ |
| 31 | 4.8*10⁵ | 1.2*10⁻³ | 2.6*10⁻⁹ |
| 32 | 2.5*10⁵ | 1.4*10⁻³ | 5.6*10⁻⁹ |
| 33 | 2.2*10⁵ | 1.5*10⁻³ | 6.7*10⁻⁹ |
| 34 | 1.2*10⁵ | 1.2*10⁻³ | 10.4*10⁻⁹ |

### INDUSTRIAL APPLICABILITY

While we have described a number of embodiments, it is apparent that our basic examples may be altered to provide other embodiments that utilize compounds and methods of the present disclosure. Therefore, it will be appreciated that the scope of an invention is to be defined by claims rather than by the specific embodiments that have been represented by way of example.

1. A CD38-binding peptide, a pharmaceutically acceptable salt thereof, or a pharmaceutically acceptable solvate thereof, wherein the CD38-binding peptide is
(1) a polypeptide having an amino acid sequence represented by SEQ ID NO. 1 or 2:
(SEQ ID NO. 1)
Ala Arg Ahp Tyr His Asp Gly Val Leu Bph Ahp Asp
Cys,
(SEQ ID NO. 2)
Ala Leu His MePhe Val Leu Pro Bph Val Trp Val Cys;
(2) a polypeptide having an amino acid sequence represented by SEQ ID NO. 1 or 2, wherein the Ala at the N-terminal is a chloroacetylated Ala;
(3) a polypeptide having an amino acid sequence with deletions, additions, substitutions or insertion of one or more amino acids in SEQ ID NO. 1 or 2, which does not comprise an amino acid sequence with deletion of Cys at the C terminal in SEQ ID NO. 1 or 2;
(4) a polypeptide having an amino acid sequence represented by SEQ ID NO. 1 or 2, wherein the Ala at the N-terminal is a chloroacetylated Ala with deletions, additions, substitutions or insertion of one or more amino acids in SEQ ID NO. 1 or 2, which does not comprise an amino acid sequence with deletion of Cys at the C terminal in SEQ ID NO. 1 or 2; or
(5) a polypeptide in accordance with one of the above (1) to (4), wherein the polypeptide has a cyclized structure.

2. The CD38-binding peptide, the pharmaceutically acceptable salt thereof, or the pharmaceutically acceptable solvate thereof in accordance with claim 1,
wherein the CD38-binding peptide has an amino acid sequence represented by one of SEQ ID NOs. 1 to 34.

3. The CD38-binding peptide, the pharmaceutically acceptable salt thereof, or the pharmaceutically acceptable solvate thereof in accordance with claim 1,
wherein the CD38-binding peptide has the cyclized structure.

4. A chemical compound comprising the CD38-binding peptide, the pharmaceutically acceptable salt thereof, or the pharmaceutically acceptable solvate thereof in accordance with claim 1,
wherein the CD38-binding peptide, the pharmaceutically acceptable salt thereof, or the pharmaceutically acceptable solvate further comprises a linker moiety.

5. The chemical compound in accordance with claim 4,
wherein the linker moiety comprises polyethylene glycol (PEG).

6. A method for treating a CD38-associated condition, disorder, or disease, comprising: administering to a subject in need thereof a pharmaceutical composition comprising
the CD38-binding peptide, the pharmaceutically acceptable salt thereof, or the pharmaceutically acceptable solvate thereof in accordance with claim 1, and
a pharmaceutically acceptable carrier.

7. The method in accordance with claim 6, wherein the CD38-associated condition, disorder, or disease is cancer.

8. The method in accordance with claim 6, wherein the CD38-associated condition, disorder, or disease is leukemia or myelomas.

9. The method in accordance with claim 6, wherein the CD38-associated condition, disorder, or disease is B-cell non-Hodgkin's Lymphoma (NHL), multiple myeloma (MM), acute myeloid leukemia (AML), acute lymphoblastic leukemia (B-cell ALL), or chronic lymphocytic leukemia (CLL).

10. The method in accordance with claim 6, wherein the CD38-associated condition, disorder, or disease is multiple myeloma (MM).

11. An agent for detecting cancer, wherein the agent comprises the CD38-binding peptide, the pharmaceutically acceptable salt thereof, or the pharmaceutically acceptable solvate thereof in accordance with claim 1.

12. The CD38-binding peptide, the pharmaceutically acceptable salt thereof, or the pharmaceutically acceptable solvate thereof in accordance with claim 2, wherein the CD38-binding peptide has the cyclized structure.
